(19) United States
(12) Patent Application Publication
(10) Pub. No.: US 2023/0374509 A1
(43) Pub. Date: Nov. 23, 2023

(54) MICRORNA INHIBITOR SYSTEM AND METHODS OF USE THEREOF

(71) Applicant: UNIVERSITY OF IOWA RESEARCH FOUNDATION, Iowa City, IA (US)

(72) Inventors: Ryan L. Boudreau, Iowa City, IA (US); Colleen S. Stein, Iowa City, IA (US); Budd Tucker, Iowa City, IA (US)

(21) Appl. No.: 18/120,204

(22) Filed: Mar. 10, 2023

Related U.S. Application Data
(60) Provisional application No. 63/318,691, filed on Mar. 10, 2022.

(51) Int. Cl. C12N 15/113; C12N 15/86; A61P 25/28; A61P 27/02

(52) U.S. Cl. CPC C12N 15/113; C12N 15/86; A61P 25/28; A61P 27/02; C12N 2750/14143

(57) ABSTRACT

The present invention relates to an expression-based RNA therapeutic that uses virus-based delivery of anti-miR181 (TuD-181). The present invention provides a method for treatment of Parkinson's disease (PD) by administering an expression-based RNA therapeutic that uses virus-based delivery of anti-miR181 (TuD-181). The present invention provides a method for treatment of retinal degeneration by administering an expression-based RNA therapeutic that uses virus-based delivery of anti-miR181 (TuD-181).

### CROSS-REFERENCE TO RELATED APPLICATION

This application claims priority to U.S. Provisional Application Number 63/318,691 that was filed on Mar. 10, 2022. The entire content of the applications referenced above is hereby incorporated by reference herein.

### SEQUENCE LISTING

The instant application contains a Sequence Listing which has been submitted electronically in XML format and is hereby incorporated by reference in its entirety. Said XML copy, created on Jun. 23, 2023, is named 17023.260US1 and is 7,609 bytes in size.

### BACKGROUND

MicroRNAs or miRNAs are short sequences of RNA (20-24 nucleotides in length) that function by altering the stability or translational efficiency of targeted mRNAs. There has been a significant amount of recent research into miRNAs that has attempted to determine their full scope, mechanism of action and disease association. Products associated with the understanding and clinical application of miRNAs will likely play a strong part in the future of medical care. Given the importance of miRs during different biological processes, tools for repression of miR function may be useful for research and have therapeutic potential. MicroRNAs are thought to regulate tumor progression and invasion via direct interaction with target genes within cells.

“Neurological disease” and “neurological disorder” refer to both hereditary and sporadic conditions that are characterized by nervous system dysfunction, and which may be associated with atrophy of the affected central or peripheral nervous system structures, or loss of function without atrophy. A neurological disease or disorder that results in atrophy is commonly called a “neurodegenerative disease” or “neurodegenerative disorder.” Neurodegenerative diseases and disorders include, but are not limited to, amyotrophic lateral sclerosis (ALS), hereditary spastic hemiplegia, primary lateral sclerosis, spinal muscular atrophy, Kennedy's disease, Alzheimer's disease, Parkinson's disease, and multiple sclerosis.

Parkinson's disease (PD) is a neurodegenerative disorder caused by progressive loss of dopaminergic (DA) neurons of the substantia nigra (SN), which project to the striatum where dopamine release stimulates neuronal circuits controlling voluntary movement. Loss of nigrostriatal dopamine results in classic motor symptoms of PD, such as tremor, rigidity, and bradykinesia. PD pathogenesis is not fully understood and mechanisms are thought to involve oxidative stress, defective autophapy/mitophagy, and neuroinflammation. Currently no treatments are available to halt PD progression.

### SUMMARY

In certain embodiments, the present invention provides an inhibitory molecule that inhibits miR-181.

In certain embodiments, the present invention provides a nucleic acid encoding an inhibitory molecule that inhibits miR-181.

In certain embodiments, the present invention provides an expression cassette comprising a nucleic acid encoding an inhibitory molecule that inhibits miR-181 operably linked to a promoter.

In certain embodiments, the present invention provides a method of inhibiting miR-181 comprising contacting a cell with a therapeutic agent, wherein the agent is an inhibitory molecule that inhibits miR-181 or the microRNA inhibitor system that inhibits miR-181, wherein the inhibitory molecule or microRNA inhibitor system reduces the level of target miR-181 by about 25% to 100%.

In certain embodiments, the present invention provides a method of treating Parkinson's disease in a patient in need thereof, comprising administering a therapeutic agent to the patient, wherein the agent is an inhibitory molecule that inhibits miR-181 or the microRNA inhibitor system that inhibits miR-181.

In certain embodiments, the present invention provides a method of treating retinal degeneration in a patient in need thereof, comprising administering a therapeutic agent to the patient, wherein the agent is an inhibitory molecule that inhibits miR-181 or the microRNA inhibitor system that inhibits miR-181.

### BRIEF DESCRIPTION OF DRAWINGS

FIG. 1. miR-181 target mRNAs are broadly depressed in aging and PD brains. Publicly-available transcriptome-wide gene expression data from studies comparing young versus old or control versus PD (or PD model, MPTP) in human and mouse brain samples were analyzed to assess mRNA levels of miR-181 targets (arrows point to lines, genes having either TargetScan-predicted 3′UTR binding sites and/or empirically-determined binding sites by Ago2 HITS-CLIP11, 15, 25), relative to all other genes (no arrows). In each of the analyzed datasets, cumulative fraction curves for miR-181 targets show leftward shifts, indicative of broad down-regulation in aging brain (e.g. 18-29 versus 65+ years-old for human and 13-26 versus 104-130 weeks-old for mice), in SN and laser-captured DA neurons from the SN pars compacta (SNpc) from PD patients, relative to non-diseased controls, and in a rodent model of PD, achieved by MPTP injection. GEO accessions (GSE#) and Kolmogorov-Smirnov (KS)-test P values are shown.

FIGS. 2A-2F. Overview and validation of miR-181 overexpression and inhibitor constructs. (FIG. 2A) Schematic of a custom AAV:miR-181 vector with a bi-cistronic cassette expressing primary miR stem-loops for miR-181a1 and miR-181b2 under control of neuronal-specific human Synapsin-1 promoter (hSYN1). (FIG. 2B) In vitro functional validation of the miR-181a1/b2 cassette was done by co-transfecting HEK293 cells with CMVmiR-181a1b2 plasmid (miR-181) or CMV-only control (Ctrl) plasmid along with a miR-181 target reporter plasmid that co-expresses a Renilla luciferase transgene with miR-181 target sites embedded within the 3′UTR, as well as a Firefly luciferase transgene for normalization. Luciferase activities were determined at 48h post-transfection and plotted as Renilla/Firefly ratios (n=3-6/group). (FIG. 2C) In vivo validation was done by direct unilateral stereotaxic injection of AAV:miR-181 (1.65e10 vg) into the SN of mice, and three weeks later, miR-181a/b and miR-29a levels were measured in RNA samples collected from SN tissues dissected from injected and contralateral non-injected control hemispheres. QPCR data were normalized to endogenous SNO135 levels and plotted as injected/non-injected ratios (n=3/group). (FIG. 2D) Schematic of a custom AAV:TuD-181 vector expressing a “tough decoy” (TuD) miR-181 inhibitor under control of the U6 promoter and a CMV-driven GFP reporter. TuD-181 harbors two high-affinity miR-181 binding sites capable of sequestering miR-181 and preventing its natural function. (FIG. 2E) In vitro functional validation of the TuD-181 cassette was done by co-transfecting N2a cells, which have low endogenous miR-181, with the luciferase-based miR-181 target reporter plasmid, synthetic miRs (miR-181a or scrambled negative control, miR-Neg), and plasmids expressing either TuD-181 or a scrambled control, TuD-Ctrl. Luciferase activities were determined at 48h post-transfection and plotted as Renilla/Firefly ratios (n=4/group), showing that TuD-181 partially blocks the ability of synthetic miR-181a to suppress the miR-181 target reporter. (FIG. 2F) In vivo validation was done by direct unilateral stereotaxic injection of either AAV:TuD-181 or AAV:GFP control (˜1.5e10 vg) into the SN of mice, and nine weeks later, endogenous miR-181a/b levels were measured in RNA samples collected from SN tissues dissected from injected or contralateral non-injected control hemispheres. QPCR data were normalized to SNO135 RNA levels and plotted as injected/non-injected ratios (n=3/group). Data in this figure are represented as the mean ±SEM. P-values were obtained using either one-way ANOVA with Tukey's multiple comparisons test (FIG. 2E) or two-tailed unpaired t-tests making the indicated comparison (FIG. 2B) or comparing non-injected versus injected sides for each miR quantified (FIG. 2C, FIG. 2F); ***p<0.001 and *p<0.05.

FIGS. 3A-3C. Characterization of an AAV-based aSyn overexpression mouse model of PD. (FIG. 3A) Schematic AAV:aSyn vectors expressing human aSyn under control of the neuronal-specific human Synapsin-1 (hSYN1) promoter with the WPRE mRNA stabilizing element. (FIG. 3B) Image analysis was performed to quantify striatal TH staining intensity in AAV:aSyn-injected and contralateral non-injected control sides (n=4 mice). P-value was obtained by two-tailed paired t-test; *p<0.05. (FIG. 3C) Elevated aSyn expression and decreased TH levels were also evident by western blot in striatal and SN tissues harvested at 9 weeks after unilateral injection of AAV:aSyn or control AAV expressing GFP via the hSYN1 promoter (AAV:GFP; 1e10 vg, n=5/group) (arrows point to results for TH). Data are represented as the mean ±SEM. All scale bars: 500 um, except for striatal images in panel B (scale bar: 200 um).

FIGS. 4A-4D. miR-181 overexpression induces TH+neuron degeneration and exacerbates aSyn-induced neurotoxicity. Histological analyses done on mouse brains injected unilaterally with AAV:U6-Ctrl (scrambled miR) or AAV:miR-181 (1.65e10 vg; harvested at 9 weeks post-injection, FIG. 4A, FIG. 4B) or co-injected unilaterally with AAV:aSyn plus or minus AAV:miR-181 (each at a low of 2.5e9 vg; harvested at 16 weeks post-injection, FIG. 4C, FIG. 4D) into the SN. Buffer-injected and AAV:GFP-injected (1e10 vg) were included as controls alongside the latter cohort. Photomicrographs of immunohistochemical staining for TH+DA neurons in striatal and SN regions were performed. Image analyses were performed to quantify striatal TH staining intensity (FIG. 4A) and TH+ neuronal cell bodies in the SN pars compacta (FIG. 4B, FIG. 4D) in injected and contralateral non-injected control sides. Plotted data are represented as the mean±SEM (n=4-5/group); P-values were obtained using either unpaired two-tailed t-tests (FIG. 4A, FIG. 4B) or one-way ANOVA with Tukey's multiple comparisons test (FIG. 4C, FIG. 4D); ***p<0.001 and *p<0.05. Scale bars: 500 um.

FIGS. 5A-5D. TuD-181 protects against aSyn-induced neurotoxicity. Histological (FIG. 5A, FIG. 5B) and western blot (FIG. 5C) analyses done on mouse brains harvested 24 weeks after bilateral stereotaxic co-injection with either AAV:GFP or AAV:aSyn (each at 1.3e10 vg) in combination with either AAV:U6-Ctrl, which expresses a scrambled stem-loop RNA control or AAV:TuD-181 (each at 1e10 vg). Photomicrographs of native GFP fluorescence or immunohistochemical staining for TH+DA neuronal cell projections and cell bodies in striatum and SN were performed. Image analyses were performed to quantify striatal TH staining intensity (FIG. 5A) and TH+neuronal cell bodies in the SN pars compacta (FIG. 5B). Plotted data are represented as the mean±SEM (n=9-10/group); P-values comparing the indicated groups were obtained using one-way ANOVA with Tukey's multiple comparisons test. The data indicate that TuD-181 significantly reduces the extent of neurodegeneration and neuronal cell loss induced by AAV:aSyn. (FIG. 5C) Western blot analyses were performed to determine if TuD-181 influences the expression of human aSyn transgene in SN or striatal tissues; no significant effect was observed (n=4/group). (FIG. 5D) Histological analyses done on mouse brains harvested 16 weeks after unilateral stereotaxic co-injection of AAV:aSyn (1.3e10 vg) in combination with either AAV:U6-Ctrl, which expresses a scrambled stem-loop RNA control or AAV:TuD-181 (each at 1e10 vg). Photomicrographs of immunohistochemical staining for TH+DA neuronal cell projections in striatum are shown for each mouse (n=5/group) were performed. Image analyses were performed to quantify striatal TH staining intensity, and ratiometric data (injected vs. non-injected hemisphere) plotted in the bar graph, represented as the mean±SEM. P-value comparing the two groups was obtained by two-tailed unpaired t-test. Scale bars: 500 μm.

FIGS. 6A-6E. Characterization of miR-181 targets in mouse SN. mRNA-sequencing was performed on SN RNA samples collected from mice three weeks after unilateral injection with AAV:miR-181 or AAV:GFP into the SN, and differential gene expression analyses were done to compare injected versus contralateral non-injected control hemispheres. (FIG. 6A) Plot showing the most significantly enriched heptamer sequences in down-regulated mRNAs (in both 3′UTR and coding sequences, CDS), based on cWords algorithm z-scores. Sequences complementary to miR-181 are highlighted in green and blue. (FIG. 6B) Cumulative fraction analysis of the gene expression data show that mRNA levels of miR-181 target genes (blue) are broadly down-regulated (i.e. leftward shifted), relative to non-target genes, red) in AAV:miR-181 injected vs. non-injected hemispheres. Kolmogorov-Smirnov (KS)-test P values is provided. (FIG. 6C) Gene ontology analyses performed on genes that were significantly down- or up-regulated after AAV:miR-181 treatment (p<0.05), but not changed in AAV:GFP samples (p>0.2). (FIG. 6D, FIG. 6E) Western blot analyses were performed using SN protein lysates harvested from mice 3 weeks after unilateral injection with either AAV:miR-181 or AAV:U6-Ctrl (scrambled miR control) to assess if select miR-181 target genes were also down-regulated at the protein level. Densitometry analysis was performed to quantify expression levels, normalized to beta-catenin loading controls. Data are represented as the mean ±SEM (n=5/group) and p-values were obtained by two-tailed unpaired t-test.

FIG. 7. Additional characterization of an AAV-based aSyn overexpression mouse model of PD. Western blot on mouse striatal and SN tissues harvested at 3 weeks after unilateral AAV:aSyn injection (1e10 vg) into SN show elevated nigral aSyn and IgG light-chain levels, while TH levels remain preserved in both regions at this early time-point. AAV:GFP injection (1e10 vg) serves as control. Representative blots are shown, and densitometry analyses were performed to quantify aSyn and TH protein levels, normalized to GAPDH loading control. Data are represented as the mean ±SEM (n=5/group) (arrows point to results for TH).

FIG. 8. Characterization of miR-181 targets in mouse SN. Western blot analyses were performed using SN protein lysates harvested from mice 3 weeks after unilateral injection with either AAV:miR-181 or AAV:GFP control to assess if select miR-181 target genes were also down-regulated at the protein level. Densitometry analysis was performed to quantify expression levels, normalized to GAPDH (Gabra1 and Chchd10) or to beta-catenin (Kcnj6) loading controls. Data are represented as the mean±SEM (n=3-5/group) and p-values obtained by one-tailed paired t-test are indicated.

FIGS. 9A-9B. SNCA expression cassette for AAV-based model. FIG. 9A. The wt human SINCA cDNA was cloned downstream of CMIV to test for protein expression after transfection into HT1080 cells. Lysates from SNICA transfected cells show strong alpha synuclein bands by western blot. FIG. 9B. Schematic of SINCA plasmid used for generating AAV5 vector.

FIG. 10. AAV5-u6-Tud-181 mediated suppression of miR-181 enhances expression of NRF1 following subretinal injection in rat.

### DETAILED DESCRIPTION

Inhibitory Molecules and Nucleic Acids Encoding Inhibitory Molecules Currently, one way to attenuate miR activity is administration of antisense oligonucleotides into cells that compete for binding with endogenous targets. A limitation of the currently used miR inhibitors resides in their inability to be retained in the tissues after cell division and they must be reapplied to maintain their effectiveness. To address these limitations and promote long-term repression of specific miRs, plasmid and/or viral vectors expressing antagomirs, sponges, eraser and Tough Decoy (TuD) RNA molecules have been reported.

MicroRNAs are small non-coding RNAs that regulate cellular gene expression. MicroRNAs play important roles in neuronal cell health and function and are dysregulated in neurodegenerative diseases, e.g. Parkinson's disease. One microRNA in particular, miR-181, was increased in PD patient brain samples. An miR-181 inhibitory molecule was developed that blocks neurodegeneration in a mouse model of PD. The current strategy employed a viral-based delivery strategy, with the recombinant virus being engineered to target neurons and express a miR-181 inhibitory RNA.

In brief, the miR-181 inhibitory RNA is expressed via the mouse U6 polIII-promoter, and this expression cassette was cloned into a AAV viral vector backbone for viral production. The design was based loosely on “Tough Decoy” (TuD) strategy for microRNA inhibition. The sequence was optimized beyond basic TuD design to ensure that the miR-181 binding sequences were better accessible and more likely to engage and sequester miR-181 from its natural targets/functions. The sequence of the mature expressed inhibitor, TuD-181, is as follows:

(SEQ ID NO: 1)
GACGGCCTCGAGACTGGCAACTCACCGACAAAGATGA
ATGTTCAAGTATTCTGGTCACAGAATACAACCTTACC
GACAAAGATGAATGTTAACACTAGTCTCGGGGCCGTC
TT

One of skill in the art would be cognizant that T's would be U's if this sequence were conveyed as RNA sequence. TuD-181 form a stem loop and presents two miR-181 binding sequences.

In certain embodiments, the present invention provides an inhibitory molecule that inhibits miR-181. In certain embodiments, the inhibitor is one of the following anti-miR sequences:

(SEQ ID NO: 2)
5′-ACUCACCGACAAAGAUGAAUGUU-3′
(SEQ ID NO: 3)
5′-CUUACCGACAAAGAUGAAUGUU-3′

In certain embodiments, the inhibitor is an LNA-based RNA to target the microRNA seed region and block the miR-181 family. In certain embodiments, the inhibitor is the following:

(SEQ ID NO: 4)
5′-GUUGAAUGUU-3′

In certain embodiments, the inhibitory molecule binds to miR-181a, miR-181b, miR-181c, and miR-181d.

In certain embodiments, the compound is RNA.

In certain embodiments, the RNA has at least 95%, 96%, 97%, 98%, 99% or 100% identity to any one of SEQ ID NO:1 (TuD-181).

In certain embodiments, a sequence is synthesized as RNA and is delivered to cells. Alternatively, in certain embodiments, commercially available or custom designed synthetic anti-mils are used to directly deliver to cells and/or tissue (e.g., brain tissue), without the need for the AAV delivery route. In certain embodiments, the present strategy provides a robust way to prevent degeneration of dopaminergic neurons by blocking mIR-181, which is known to promote cell death pathways.

As used herein, the term “nucleic acid” and “polynucleotide” refers to deoxyribonucleotides or ribonucleotides and polymers thereof in either single- or double-stranded form, composed of monomers (nucleotides) containing a sugar, phosphate and a base that is either a purine or pyrimidine. Unless specifically limited, the term encompasses nucleic acids containing known analogs of natural nucleotides which have similar binding properties as the reference nucleic acid and are metabolized in a manner similar to naturally occurring nucleotides

“Operably-linked” refers to the association two chemical moieties so that the function of one is affected by the other, e.g., an arrangement of elements wherein the components so described are configured so as to perform their usual functions.

### Expression Cassettes

“Expression cassette” as used herein means a nucleic acid sequence capable of directing expression of a particular nucleotide sequence in an appropriate host cell, which includes a promoter operably linked to the nucleotide sequence of interest that may be operably linked to termination signals. The coding region usually codes for a functional RNA of interest, for example an siRNA. The expression cassette including the nucleotide sequence of interest may be chimeric. The expression cassette may also be one that is naturally occurring but has been obtained in a recombinant form useful for heterologous expression. The expression of the nucleotide sequence in the expression cassette may be under the control of a constitutive promoter or of a regulatable promoter that initiates transcription only when the host cell is exposed to some particular stimulus. In the case of a multicellular organism, the promoter can also be specific to a particular tissue or organ or stage of development.

Such expression cassettes can include a transcriptional initiation region linked to a nucleotide sequence of interest. Such an expression cassette is provided with a plurality of restriction sites for insertion of the gene of interest to be under the transcriptional regulation of the regulatory regions. The expression cassette may additionally contain selectable marker genes.

“Antisense inhibition” refers to the production of antisense RNA transcripts capable of suppressing the expression of protein from an endogenous gene or a transgene.

To prepare expression cassettes, the recombinant DNA sequence or segment may be circular or linear, double-stranded or single-stranded. Generally, the DNA sequence or segment is in the form of chimeric DNA, such as plasmid DNA or a vector that can also contain coding regions flanked by control sequences that promote the expression of the recombinant DNA present in the resultant transformed cell.

A “chimeric” vector or expression cassette, as used herein, means a vector or cassette including nucleic acid sequences from at least two different species, or has a nucleic acid sequence from the same species that is linked or associated in a manner that does not occur in the “native” or wild type of the species.

Aside from recombinant DNA sequences that serve as transcription units for an RNA transcript, or portions thereof, a portion of the recombinant DNA may be untranscribed, serving a regulatory or a structural function. For example, the recombinant DNA may have a promoter that is active in mammalian cells.

Other elements functional in the host cells, such as introns, enhancers, polyadenylation sequences and the like, may also be a part of the recombinant DNA. Such elements may or may not be necessary for the function of the DNA, but may provide improved expression of the DNA by affecting transcription, stability of the siRNA, or the like. Such elements may be included in the DNA as desired to obtain the optimal performance of the siRNA in the cell.

Control sequences are DNA sequences necessary for the expression of an operably linked coding sequence in a particular host organism. The control sequences that are suitable for prokaryotic cells, for example, include a promoter, and optionally an operator sequence, and a ribosome binding site. Eukaryotic cells are known to utilize promoters, polyadenylation signals, and enhancers.

Operably linked nucleic acids are nucleic acids placed in a functional relationship with another nucleic acid sequence. For example, a promoter or enhancer is operably linked to a coding sequence if it affects the transcription of the sequence; or a ribosome binding site is operably linked to a coding sequence if it is positioned so as to facilitate translation. Generally, operably linked DNA sequences are DNA sequences that are linked are contiguous. However, enhancers do not have to be contiguous. Linking is accomplished by ligation at convenient restriction sites. If such sites do not exist, the synthetic oligonucleotide adaptors or linkers are used in accord with conventional practice.

The recombinant DNA to be introduced into the cells may contain either a selectable marker gene or a reporter gene or both to facilitate identification and selection of expressing cells from the population of cells sought to be transfected or infected through viral vectors. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neo and the like.

Reporter genes are used for identifying potentially transfected cells and for evaluating the functionality of regulatory sequences. Reporter genes that encode for easily assayable proteins are well known in the art. In general, a reporter gene is a gene that is not present in or expressed by the recipient organism or tissue and that encodes a protein whose expression is manifested by some easily detectable property, e.g., enzymatic activity. For example, reporter genes include the chloramphenicol acetyl transferase gene (cat) from Tn9 of E. coli and the luciferase gene from firefly Photinus pyrahs. Expression of the reporter gene is assayed at a suitable time after the DNA has been introduced into the recipient cells.

The general methods for constructing recombinant DNA that can transfect target cells are well known to those skilled in the art, and the same compositions and methods of construction may be utilized to produce the DNA useful herein.

The recombinant DNA can be readily introduced into the host cells, e.g., mammalian, bacterial, yeast or insect cells by transfection with an expression vector composed of DNA encoding the siRNA by any procedure useful for the introduction into a particular cell, e.g., physical or biological methods, to yield a cell having the recombinant DNA stably integrated into its genome or existing as a episomal element, so that the DNA molecules, or sequences of the present invention are expressed by the host cell. Preferably, the DNA is introduced into host cells via a vector. The host cell is preferably of eukaryotic origin, e.g., plant, mammalian, insect, yeast or fungal sources, but host cells of non-eukaryotic origin may also be employed. The described miR Inhibitor can also be introduced into host cells as an in vitro transcribed RNA molecule, without the use of a vector. This miR Inhibitor RNA molecule works exactly like the Plasmid-Based miR Inhibitor to inhibit microRNA function.

Physical methods to introduce a preselected DNA into a host cell include calcium phosphate precipitation, lipofection, particle bombardment, microinjection, electroporation, nanoparticles and the like. Biological methods to introduce the DNA of interest into a host cell include the use of DNA and RNA viral vectors. For mammalian gene therapy, as described herein below, it is desirable to use an efficient means of inserting a copy gene into the host genome. Viral vectors, and especially retroviral vectors, have become the most widely used method for inserting genes into mammalian, e.g., human cells. Other viral vectors can be derived from poxviruses, herpes simplex virus I, adenoviruses and adeno-associated viruses, and the like. See, for example, U.S. Pat. Nos. 5,350,674 and 5,585,362.

As discussed herein, a “transfected” or “transduced” host cell or cell line is one in which the genome has been altered or augmented by the presence of at least one heterologous or recombinant nucleic acid sequence. The host cells of the present invention are typically produced by transfection with a DNA sequence in a plasmid expression vector, a viral expression vector, or as an isolated linear DNA sequence. The transfected DNA can become a chromosomally integrated recombinant DNA sequence, which is composed of sequence encoding the siRNA.

To confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

To detect and quantitate RNA produced from introduced recombinant DNA segments, RT-PCR and/or Northern blotting may be employed. In this application of PCR, it is first necessary to reverse transcribe RNA into DNA, using enzymes such as reverse transcriptase, and then through the use of conventional PCR techniques amplify the DNA. In most instances PCR techniques, while useful, will not demonstrate integrity of the RNA product. Further information about the nature of the RNA product may be obtained by Northern blotting. This technique demonstrates the presence of an RNA species and gives information about the integrity of that RNA. The presence or absence of an RNA species can also be determined using dot or slot blot Northern hybridizations. These techniques are modifications of Northern blotting and only demonstrate the presence or absence of an RNA species.

While Southern blotting and PCR may be used to detect the recombinant DNA segment in question, they do not provide information as to whether the preselected DNA segment is being expressed. Expression may be evaluated by specifically identifying the peptide products of the introduced recombinant DNA sequences or evaluating the phenotypic changes brought about by the expression of the introduced recombinant DNA segment in the host cell.

The instant invention provides a cell expression system for expressing exogenous nucleic acid material in a mammalian recipient. The expression system, also referred to as a “genetically modified cell,” comprises a cell and an expression vector for expressing the exogenous nucleic acid material. The genetically modified cells are suitable for administration to a mammalian recipient, where they replace the endogenous cells of the recipient. Thus, the preferred genetically modified cells are non-immortalized and are non-tumorigenic.

According to one embodiment, the cells are transfected or otherwise genetically modified ex vivo. The cells are isolated from a mammal (preferably a human), nucleic acid introduced (i.e., transduced or transfected in vitro) with a vector for expressing a heterologous (e.g., recombinant) gene encoding the therapeutic agent, and then administered to a mammalian recipient for delivery of the therapeutic agent in situ. The mammalian recipient may be a human and the cells to be modified are autologous cells, i.e., the cells are isolated from the mammalian recipient.

According to another embodiment, the cells are transfected or transduced or otherwise genetically modified in vivo. The cells from the mammalian recipient are transduced or transfected in vivo with a vector containing exogenous nucleic acid material for expressing a heterologous (e.g., recombinant) gene encoding a therapeutic agent and the therapeutic agent is delivered in situ.

As used herein, “exogenous nucleic acid material” refers to a nucleic acid or an oligonucleotide, either natural or synthetic, which is not naturally found in the cells; or if it is naturally found in the cells, is modified from its original or native form. Thus, “exogenous nucleic acid material” includes, for example, a non-naturally occurring nucleic acid that can be transcribed into a miR inhibitor.

### Promoters

The present invention further provides an expression cassette containing a promoter contiguously linked to a nucleic acid described herein. In certain embodiments, the promoter is a polII or a polIII promoter, such as a U6 promoter (e.g., a mouse U6 promoter). In certain embodiments, the expression cassette further contains a marker gene. In certain embodiments, the promoter is a polII promoter. In certain embodiments, the promoter is a tissue-specific promoter. In certain embodiments, the promoter is an inducible promoter. In certain embodiments, the promoter is a polIII promoter. In certain embodiments, the promoter is an inducible promoter. In certain embodiments, the expression cassette uses a constitutive promoter, tissue-specific promotes, development-specific promotes, regulatable promoter or viral promoter.

“Promoter” refers to a nucleotide sequence, usually upstream (5′) to its coding sequence, which directs and/or controls the expression of the coding sequence by providing the recognition for RNA polymerase and other factors required for proper transcription. “Promoter” includes a minimal promoter that is a short DNA sequence comprised of a TATA- box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence or functional RNA. This type of promoter sequence consists of proximal and more distal upstream elements, the latter elements often referred to as enhancers. Accordingly, an “enhancer” is a DNA sequence that can stimulate promoter activity and may be an innate element of the promoter or a heterologous element inserted to enhance the level or tissue specificity of a promoter. It is capable of operating in both orientations (normal or flipped), and is capable of functioning even when moved either upstream or downstream from the promoter. Both enhancers and other upstream promoter elements bind sequence-specific DNA-binding proteins that mediate their effects. Promoters may be derived in their entirety from a native gene, or be composed of different elements derived from different promoters found in nature, or even be comprised of synthetic DNA segments. A promoter may also contain DNA sequences that are involved in the binding of protein factors that control the effectiveness of transcription initiation in response to physiological or developmental conditions. Examples of promoters that may be used in the present invention include the mouse U6 RNA promoters, synthetic human H1RNA promoters, SV40, CMV, RSV, RNA polymerase II and RNA polymerase III promoters.

The “initiation site” is the position surrounding the first nucleotide that is part of the transcribed sequence, which is also defined as position +1. With respect to this site all other sequences of the gene and its controlling regions are numbered. Downstream sequences (i.e., further protein encoding sequences in the 3′ direction) are denominated positive, while upstream sequences (mostly of the controlling regions in the 5′ direction) are denominated negative.

Promoter elements, particularly a TATA element, that are inactive or that have greatly reduced promoter activity in the absence of upstream activation are referred to as “minimal or core promoters.” In the presence of a suitable transcription factor, the minimal promoter functions to permit transcription. A “minimal or core promoter” thus consists only of all basal elements needed for transcription initiation, e.g., a TATA box and/or an initiator.

“Constitutive expression” refers to expression using a constitutive or regulated promoter. “Conditional” and “regulated expression” refer to expression controlled by a regulated promoter.

“Operably-linked” refers to the association of nucleic acid sequences on single nucleic acid fragment so that the function of one of the sequences is affected by another. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation.

“Expression” refers to the transcription and/or translation of an endogenous gene, heterologous gene or nucleic acid segment, or a transgene in cells. For example, in the case of siRNA constructs, expression may refer to the transcription of the siRNA only. In addition, expression refers to the transcription and stable accumulation of sense (mRNA) or functional RNA. Expression may also refer to the production of protein.

In certain embodiments, the promoter is a transiently expressed or is constitutively expressed.

In certain embodiments, the promoter is a tissue-specific or inducible promoter.

In certain embodiments, the promoter is a mouse U6 polIII promoter.

### Vectors

In certain embodiments, the vector is in the form of a plasmid, which is transferred into the target cells by one of a variety of methods: physical (e.g., microinjection, electroporation, scrape loading, microparticle bombardment) or by cellular uptake as a chemical complex (e.g., calcium or strontium co-precipitation, complexation with lipid, complexation with ligand). Several commercial products are available for cationic liposome complexation including Lipofectin™ (Gibco-BRL, Gaithersburg, Md.) and Transfectam™ (Promega®, Madison, Wis.). However, the efficiency of transfection by these methods is highly dependent on the nature of the target cell and accordingly, the conditions for optimal transfection of nucleic acids into cells using the herein-mentioned procedures must be optimized. Such optimization is within the scope of one of ordinary skill in the art without the need for undue experimentation. In certain embodiments, the vector is pSilencer 4.1 (Ambion).

In certain embodiments, the vector is an adenoviral, lentiviral, adeno-associated viral (AAV), poliovirus, HSV, or murine Maloney-based viral vector. In certain embodiments, the vector is pLL3.7 vector. In certain embodiments, the vector is any Polymerase II or III expression vector and/or transgenic vector.

In certain embodiments, the vector is an adeno-associated virus (AAV). In certain embodiments, the vector is a neurotropic AAV. In certain embodiments, the AAV is AAV2/5, AAV2/1, AAV2/9, PHP.B, or PHP.eB.

### Nucleic Acid Molecules

The terms “isolated and/or purified” refer to in vitro isolation of a nucleic acid, e.g., a DNA or RNA molecule from its natural cellular environment, and from association with other components of the cell, such as nucleic acid or polypeptide, so that it can be sequenced, replicated, and/or expressed. The RNA or DNA is “isolated” in that it is free from at least one contaminating nucleic acid with which it is normally associated in the natural source of the RNA or DNA and is preferably substantially free of any other mammalian RNA or DNA. The phrase “free from at least one contaminating source nucleic acid with which it is normally associated” includes the case where the nucleic acid is reintroduced into the source or natural cell but is in a different chromosomal location or is otherwise flanked by nucleic acid sequences not normally found in the source cell, e.g., in a vector or plasmid.

As used herein, the term “recombinant nucleic acid”, e.g., “recombinant DNA sequence or segment” refers to a nucleic acid, e.g., to DNA, that has been derived or isolated from any appropriate cellular source, that may be subsequently chemically altered in vitro, so that its sequence is not naturally occurring, or corresponds to naturally occurring sequences that are not positioned as they would be positioned in a genome which has not been transformed with exogenous DNA. An example of preselected DNA “derived” from a source would be a DNA sequence that is identified as a useful fragment within a given organism, and which is then chemically synthesized in essentially pure form. An example of such DNA “isolated” from a source would be a useful DNA sequence that is excised or removed from a source by chemical means, e.g., by the use of restriction endonucleases, so that it can be further manipulated, e.g., amplified, for use in the invention, by the methodology of genetic engineering. “Recombinant DNA” includes completely synthetic DNA sequences, semi-synthetic DNA sequences, DNA sequences isolated from biological sources, and DNA sequences derived from RNA, as well as mixtures thereof.

### Methods for Introducing the Expression Cassettes of the Invention into Cells

The inhibitory nucleic acid material (e.g., an expression cassette encoding a miR inhibitor) can be introduced into the cell ex vivo or in vivo by genetic transfer methods, such as transfection or transduction, to provide a genetically modified cell. Various expression vectors (i.e., vehicles for facilitating delivery of exogenous nucleic acid into a target cell) are known to one of ordinary skill in the art.

As used herein, “transfection of cells” refers to the acquisition by a cell of new nucleic acid material by incorporation of added DNA. Thus, transfection refers to the insertion of nucleic acid into a cell using physical or chemical methods. Several transfection techniques are known to those of ordinary skill in the art including calcium phosphate DNA co-precipitation, DEAE-dextran, electroporation, nanoparticles, cationic liposome-mediated transfection, tungsten particle-facilitated microparticle bombardment, and strontium phosphate DNA co-precipitation.

In contrast, “transduction of cells” refers to the process of transferring nucleic acid into a cell using a DNA or RNA virus. A RNA virus (i.e., a retrovirus) for transferring a nucleic acid into a cell is referred to herein as a transducing chimeric retrovirus. Exogenous nucleic acid material contained within the retrovirus is incorporated into the genome of the transduced cell. A cell that has been transduced with a chimeric DNA virus (e.g., an adenovirus carrying a cDNA encoding a therapeutic agent), will not have the exogenous nucleic acid material incorporated into its genome but will be capable of expressing the exogenous nucleic acid material that is retained extrachromosomally within the cell.

The exogenous nucleic acid material can include the nucleic acid encoding the miR inhibitor together with a promoter to control transcription. The promoter characteristically has a specific nucleotide sequence necessary to initiate transcription. The exogenous nucleic acid material may further include additional sequences (i.e., enhancers) required to obtain the desired transcription activity. For the purpose of this discussion an “enhancer” is simply any non-translated DNA sequence that works with the coding sequence (in cis) to change the basal transcription level dictated by the promoter. The exogenous nucleic acid material may be introduced into the cell genome immediately downstream from the promoter so that the promoter and coding sequence are operatively linked so as to permit transcription of the coding sequence. An expression vector can include an exogenous promoter element to control transcription of the inserted exogenous gene. Such exogenous promoters include both constitutive and regulatable promoters.

Naturally-occurring constitutive promoters control the expression of essential cell functions. As a result, a nucleic acid sequence under the control of a constitutive promoter is expressed under all conditions of cell growth. Constitutive promoters include the promoters for the following genes which encode certain constitutive or “housekeeping” functions: hypoxanthine phosphoribosyl transferase (HPRT), dihydrofolate reductase (DHFR), adenosine deaminase, phosphoglycerol kinase (PGK), pyruvate kinase, phosphoglycerol mutase, the beta-actin promoter, and other constitutive promoters known to those of skill in the art. In addition, many viral promoters function constitutively in eukaryotic cells. These include: the early and late promoters of SV40; the long terminal repeats (LTRs) of Moloney Leukemia Virus and other retroviruses; and the thymidine kinase promoter of Herpes Simplex Virus, among many others.

Nucleic acid sequences that are under the control of regulatable promoters are expressed only or to a greater or lesser degree in the presence of an inducing or repressing agent, (e.g., transcription under control of the metallothionein promoter is greatly increased in presence of certain metal ions). Regulatable promoters include responsive elements (REs) that stimulate transcription when their inducing factors are bound. For example, there are REs for serum factors, steroid hormones, retinoic acid, cyclic AMP, and tetracycline and doxycycline. Promoters containing a particular RE can be chosen in order to obtain an regulatable response and in some cases, the RE itself may be attached to a different promoter, thereby conferring regulatability to the encoded nucleic acid sequence. Thus, by selecting the appropriate promoter (constitutive versus regulatable; strong versus weak), it is possible to control both the existence and level of expression of a nucleic acid sequence in the genetically modified cell. If the nucleic acid sequence is under the control of an regulatable promoter, delivery of the therapeutic agent in situ is triggered by exposing the genetically modified cell in situ to conditions for permitting transcription of the nucleic acid sequence, e.g., by intraperitoneal injection of specific inducers of the regulatable promoters which control transcription of the agent. For example, in situ expression of a nucleic acid sequence under the control of the metallothionein promoter in genetically modified cells is enhanced by contacting the genetically modified cells with a solution containing the appropriate (i.e., inducing) metal ions in situ.

Accordingly, the amount of miR inhibitor generated in situ is regulated by controlling such factors as the nature of the promoter used to direct transcription of the nucleic acid sequence, (i.e., whether the promoter is constitutive or regulatable, strong or weak) and the number of copies of the exogenous nucleic acid sequence encoding a miR inhibitor sequence that are in the cell.

In addition to at least one promoter and at least one heterologous nucleic acid sequence encoding the miR inhibitor, the expression vector may include a selection gene, for example, a neomycin resistance gene, for facilitating selection of cells that have been transfected or transduced with the expression vector.

Cells can also be transfected with two or more expression vectors, at least one vector containing the nucleic acid sequence(s) encoding the miR inhibitor (s), the other vector containing a selection gene. The selection of a suitable promoter, enhancer, selection gene, and/or signal sequence is deemed to be within the scope of one of ordinary skill in the art without undue experimentation.

The following discussion is directed to various utilities of the instant invention. For example, the instant invention has utility as an expression system suitable for silencing the nucleic acid sequence of interest.

The instant invention also provides methods for genetically modifying cells of a mammalian recipient in vivo. According to one embodiment, the method comprises introducing an expression vector for expressing a miR inhibitor sequence in cells of the mammalian recipient in situ by, for example, injecting the vector into the recipient.

### Delivery Systems for Delivering Microrna Inhibitors to Human and Murine Cells and Tissues

A wide range of nano-sized complexes, nanoparticles, microparticles and lipid based delivery systems are used to deliver microRNA Inhibitors to human and murine cells and tissues. These include synthetic cationic polymers such as polyethylenimine and natural polymers such as chitosan that can form complexes with the microRNA inhibitors. The microRNA inhibitors can be loaded into cationic, anionic and neutral liposomes. Also, the microRNA inhibitors can be loaded into biodegradable synthetic polymers such as polylactide-co-glycolide (PLGA), PLA, polycaprolactone (PCL), polyanhydrides (PA). This list of provided materials is not exhaustive and we often use combinations and permutations of these materials such as preparing PLGA and PEI. A wide range of cell binding or cell targeting ligands can be conjugated to these delivery systems including (but not limited to) transferrin, cell penetrating peptides like RGD or TAT, aptamers, galactose and mannose.

A polymeric microparticle core as described herein can comprise one or more polymers. Polymers can be selected from the group consisting of biocompatible and/or biodegradable polymers. As used herein, the term “biodegradable” refers to the ability of a composition to erode or degrade in vivo to form smaller chemical fragments. Degradation may occur, for example, by enzymatic, chemical or physical processes. Non-limiting examples of biodegradable polymers that can be used in aspects of the invention include poly(lactide)s, poly(glycolide)s, poly(lactic acid)s, poly(glycolic acid)s, poly (lactide-co-glycolide), polyanhydrides, polyorthoesters, polycaprolactone, polyesteramides, polycarbonate, polycyanoacrylate, polyurethanes, polyacrylate, blends and copolymers thereof.

Other additional biodegradable polymers include biodegradable polyetherester copolymers. Generally, the polyetherester copolymers are amphiphilic block copolymers that include hydrophilic (for example, a polyalkylene glycol, such as polyethylene glycol) and hydrophobic blocks (for example, polyethylene terephthalate). An exemplary block copolymer is, but is not limited to, poly(ethylene glycol)-based and poly(butylene terephthalate)-based blocks (PEG/PBT polymer) and PLGA. PEG/PBT polymers are commercially available from OctoPlus Inc, under the trade designation PolyActive™. Non-limiting examples of biodegradable copolymers or multiblock copolymers include the ones described in U.S. Pat. Nos: 5,980,948 and 5,252,701, the contents of which are incorporated herein by reference in their entirety.

Other biodegradable polymer materials include biodegradable terephthalate copolymers that include a phosphorus-containing linkage. Polymers having phosphoester linkages, called poly(phosphates), poly(phosphonates) and poly(phosphites), are known in the art.

Biodegradable polyhydric alcohol esters can also be used for the purposes of the invention. In some embodiments, the biodegradable polymer can be a three-dimensional crosslinked polymer network containing hydrophobic and hydrophilic components that form a hydrogel with a crosslinked polymer structure, such as the one described in U.S. Pat. No. 6,583,219. In yet further embodiments, the biodegradable polymer can comprise a polymer based upon α-amino acids (such as elastomeric copolyester amides or copolyester urethanes, as described in U.S. Pat. No. 6,503,538, which is incorporated herein by reference in its entirety).

In one embodiment, the polymeric microparticle core described herein comprises poly(lactide-co-glycolide) (PLGA). In certain embodiments, the polymeric microparticle core described herein comprises at least about 20%, at least about 30%, at least about 40%, at least about 50%, at least about 60%, at least about 70%, at least about 80%, at least about 90%, at least about 95%, about 98%, about 99% or 100% of PLGA.

In general, any biocompatible material well known in the art for fabrication of microparticles can be used in embodiments of the microparticle described herein. Accordingly, a microparticle comprising a lipidic microparticle core is also within the scope of the invention. An exemplary lipidic microparticle core is, but is not limited to, a liposome. A liposome is generally defined as a particle comprising one or more lipid bilayers enclosing an interior, e.g., an aqueous interior. In one embodiment, a liposome can be a vesicle formed by a bilayer lipid membrane. Methods for the preparation of liposomes are well described in the art.

The cationic dendrimer as described herein is generally a repeatedly branched and roughly spherical molecule with one or more positively-charged functional groups. In one embodiment, the cationic dendrimer is symmetric around the core, and generally adopts a roughly spherical three-dimensional morphology. In a particular embodiment, the cationic dendrimer used for surface modification of the microparticle core is poly(amidoamine) or PAMAM. The core of PAMAM is a diamine (commonly ethylenediamine), which is reacted with methyl acrylate, and then another ethylenediamine to make the generation-0 (G-0) PAMAM. Successive reactions create higher generations, which tend to have different properties. Lower generations can be considered as flexible molecules with no appreciable inner regions, while medium sized (G-3 or G-4) can have internal space that is essentially separated from the outer shell of the dendrimer. Very large (G-7 and greater) dendrimers can be considered as roughly solid particles with very dense surfaces due to the structure of their outer shell. In one embodiment, the outer surface of the microparticle core is modified with PAMAM Generation-3.

Without limitations, in some embodiments, other positively-charged polymer molecules can also be used to modify the outer surface of the microparticle core described herein. Examples of positively-charged polymers include, but are not limited to, polyamino acids such as polylysine, polyhistidine, polyornithine, polycitrulline, polyhydroxylysine, polyarginine, polyhomoarginine, polyaminotyrosine, and protamines. Other suitable positively-charged polymers include, but are not limited to, polydiaminobutyric acid, polyethyleneimine, polypropyleneimine, polyamino(meth)acrylate, polyaminostyrene, polyaminoethylene, poly(aminoethyl)ethylene, polyaminoethylstyrene, diethyl amino ethyl cellulose, poly-imino tyrosine, cholestyramine-resin, poly-imino acid, 1,5-dimethyl-1,5-diazaundecamethylene polymethobromide (hexadimethrine bromide), chitosan, poly(amidoamine) dendrimers, and combinations thereof.

### Methods of Treatment

In certain embodiments, the present invention provides a method of inhibiting miR-181 comprising contacting a cell with a therapeutic agent, wherein the agent is an inhibitory molecule that inhibits miR-181 or the microRNA inhibitor system that inhibits miR-181, wherein the inhibitory molecule or microRNA inhibitor system reduces the level of target miR-181 by about 25% to 100%.

In certain embodiments, the therapeutic agent reduces the level of target miR by about 90%.

In certain embodiments, the present invention provides a method of treating Parkinson's disease in a patient in need thereof, comprising administering a therapeutic agent to the patient, wherein the therapeutic agent is the inhibitory molecule or the microRNA inhibitor system that inhibits miR-181.

In certain embodiments, the therapeutic agent is the inhibitory molecule that inhibits miR-181, and the therapeutic agent is administered to the cerebrospinal fluid.

In certain embodiments, the therapeutic agent is the microRNA inhibitor system that inhibits miR-181, and the therapeutic agent is administered locally in the brain. In certain embodiments, the microRNA inhibitor is administered intravenously. In certain embodiments, the microRNA inhibitor is delivered via the spinal fluid.

In certain embodiments, the therapeutic agent suppresses at least 10% of miR-181 activity.

In certain embodiments, the therapeutic agent suppresses at least 25% of miR-181 activity.

In certain embodiments, the therapeutic agent suppresses at least 50% of miR-181 activity.

In certain embodiments, the therapeutic agent suppresses at least 60% of miR-181 activity.

In certain embodiments, the present invention provides an inhibitory molecule that inhibits miR-181. In certain embodiments, the inhibitor is one of the following anti-miR sequences:

(SEQ ID NO: 2)
5′-ACUCACCGACAAAGAUGAAUGUU-3′
(SEQ ID NO: 3)
5′-CUUACCGACAAAGAUGAAUGUU-3′

In certain embodiments, the inhibitor is an LNA-based RNA to target the microRNA seed region and block the miR-181 family. In certain embodiments, the inhibitor is the following:

(SEQ ID NO: 4)
5′-GUUGAAUGUU-3′

### Dosages, Formulations and Routes of Administration of the Agents of the Invention

The agents of the invention are preferably administered so as to result in a reduction in at least one symptom associated with a disease. The amount administered will vary depending on various factors including, but not limited to, the composition chosen, the particular disease, the weight, the physical condition, and the age of the mammal, and whether prevention or treatment is to be achieved. Such factors can be readily determined by the clinician employing animal models or other test systems, which are well known to the art. As used herein, the term “therapeutic miR inhibitor” refers to any siRNA that has a beneficial effect on the recipient. Thus, “therapeutic miR inhibitor” embraces both therapeutic and prophylactic miR inhibitor.

Administration of miR inhibitor may be accomplished through the administration of the nucleic acid molecule encoding the siRNA. Pharmaceutical formulations, dosages and routes of administration for nucleic acids are generally known.

One or more suitable unit dosage forms having the therapeutic agent(s) of the invention, which, as discussed below, may optionally be formulated for sustained release (for example using microencapsulation), can be administered by a variety of routes including parenteral, including by intravenous and intramuscular routes, as well as by direct injection into the diseased tissue. For example, the therapeutic agent may be directly injected into the brain. Alternatively the therapeutic agent may be introduced intrathecally for brain and spinal cord conditions. In another example, the therapeutic agent may be introduced intramuscularly for viruses that traffic back to affected neurons from muscle, such as AAV, lentivirus and adenovirus. The formulations may, where appropriate, be conveniently presented in discrete unit dosage forms and may be prepared by any of the methods well known to pharmacy. Such methods may include the step of bringing into association the therapeutic agent with liquid carriers, solid matrices, semi-solid carriers, finely divided solid carriers or combinations thereof, and then, if necessary, introducing or shaping the product into the desired delivery system.

When the therapeutic agents of the invention are prepared for administration, they are preferably combined with a pharmaceutically acceptable carrier, diluent or excipient to form a pharmaceutical formulation, or unit dosage form. The total active ingredients in such formulations include from 0.1 to 99.9% by weight of the formulation. A “pharmaceutically acceptable” is a carrier, diluent, excipient, and/or salt that is compatible with the other ingredients of the formulation, and not deleterious to the recipient thereof. The active ingredient for administration may be present as a powder or as granules, as a solution, a suspension or an emulsion.

Pharmaceutical formulations containing the therapeutic agents of the invention can be prepared by procedures known in the art using well known and readily available ingredients. The therapeutic agents of the invention can also be formulated as solutions appropriate for parenteral administration, for instance by intramuscular, subcutaneous or intravenous routes.

The pharmaceutical formulations of the therapeutic agents of the invention can also take the form of an aqueous or anhydrous solution or dispersion, or alternatively the form of an emulsion or suspension.

Thus, the therapeutic agent may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

It will be appreciated that the unit content of active ingredient or ingredients contained in an individual aerosol dose of each dosage form need not in itself constitute an effective amount for treating the particular indication or disease since the necessary effective amount can be reached by administration of a plurality of dosage units. Moreover, the effective amount may be achieved using less than the dose in the dosage form, either individually, or in a series of administrations.

The pharmaceutical formulations of the present invention may include, as optional ingredients, pharmaceutically acceptable carriers, diluents, solubilizing or emulsifying agents, and salts of the type that are well-known in the art. Specific non-limiting examples of the carriers and/or diluents that are useful in the pharmaceutical formulations of the present invention include water and physiologically acceptable buffered saline solutions such as phosphate buffered saline solutions pH 7.0-8.0. saline solutions and water.

The invention will now be illustrated by the following non-limiting Example.

In vivo mammalian introduction of the miR Inhibitor DNA into the genome of a species can be accomplished using gene targeting systems. The miR Inhibitor can be inserted into the genome of any animal using the ROSA 26 loci for either continuous or induced expression of the miR Inhibitor. In additional a transgenic animal can be produced expressing the miR Inhbitor by pronuclear injection and insertion into nonspecific chromatin. Additional gene can be linked to the expression of the miR Inhibitor in animals.

### EXAMPLE 1

### Modulation of miR-181 Influences Dopaminergic Neuronal Degeneration in a Mouse Model of Parkinson'S Disease

Parkinson's disease (PD) is caused by loss of dopaminergic (DA) neurons in the substantia nigra (SN). Although PD pathogenesis is not fully understood, studies implicate perturbations in gene regulation, mitochondrial function, and neuronal activity. MicroRNAs (miRs) are small gene regulatory RNAs that inhibit diverse subsets of target mRNAs, and several studies have noted miR expression alterations in PD brains. For example, miR-181a is abundant in brain and is increased in PD patient brain samples; however, the disease relevance of this remains unclear. Herein, we show that miR-181 target mRNAs are broadly down-regulated in aging and PD brains. To address if the miR-181 family plays a role in PD pathogenesis, we generated adeno-associated viruses (AAV) to overexpress and inhibit miR-181 isoforms. After co-injection with AAV overexpressing alpha-synuclein (aSyn) into mouse SN (PD model), we found that moderate miR-181a/b overexpression exacerbated aSyn-induced DA neuronal loss, whereas miR-181 inhibition was neuroprotective, relative to controls (GFP-alone and/or scrambled RNA). Also, prolonged miR-181 overexpression in SN alone elicited measurable neurotoxicity coincident with an increased immune response. RNA-seq analyses revealed that miR-181a/b inhibits genes involved in synaptic transmission, neurite outgrowth, and mitochondrial respiration, along with several genes having known protective roles and genetic links in PD.

### Introduction

Parkinson's disease (PD) is the second most prevalent aging-related neurodegenerative disease worldwide, affecting ˜10 million individuals, the vast majority of whom are diagnosed with sporadic PD. PD patients suffer progressive loss of dopaminergic (DA) neurons that reside in the substantia nigra (SN) and project to and release dopamine in the striatum. Disease progression coincides with the hallmark accumulation of deposited protein aggregates known as Lewy bodies. The primary component of Lewy bodies is alpha-synuclein (aSyn), a protein with poorly understood function that is often elevated in PD patient brains. Indeed, overexpression of aSyn can elicit the onset of PD and neuronal dysfunction in humans (e.g., individuals with triplication of the gene encoding aSyn1, SNCA), as well as in mice (e.g., aSyn transgenic mouse models of PD). In addition, several groups have demonstrated that viral-mediated aSyn overexpression by direct injection of recombinant adeno-associated virus (AAV) into rodent brains serves as a valuable model of PD that recapitulates many disease aspects (e.g., aSyn aggregation, DA neuronal degeneration, neuroinflammation, and in some cases, motor deficits). However, the precise mechanisms of aSyn-induced neurotoxicity are not fully understood, and this has hindered therapeutic development for PD.

Mechanistic studies have implicated several important biological pathways/processes in PD pathogenesis, including impaired lysosomal-autophagy pathways, calcium overload and mitochondrial dysfunction. Moreover, aberrations in transcriptional and post-transcriptional gene regulation are evident in PD mouse models and in post-mortem PD patient brains. For example, several groups have done small RNA profiling in PD brain samples to identify changes in microRNAs (miRs), which serve as master regulators of the transcriptome and play critical roles in nervous system biology. The human genome encodes for ˜2000 miRs, which are non-coding RNAs that incorporate into the Argonaute (Ago)-RISC silencing complexes to direct target mRNA silencing (e.g., transcript destabilization) typically through miR-mRNA base-pairing via the seed region (positions 2-8 of the miR). These RNAs are capable of regulating a diverse set of target mRNAs to uniquely coordinate a biological response, and their potential to serve as therapeutic targets is being investigated for a broad range of diseases.

While exploring the literature and related available datasets, we noted multiple instances in which miR-181a levels were found to be elevated in aging brain tissues, as well as in PD brains and cerebrospinal fluid, with one study showing a positive correlation with Lewy body deposition. MiR-181a is part of the miR-181a/b/c/d family, which exhibits perfect sequence conservation among most species (including humans and rodents) and is predicted to engage many highly-conserved target sites within shared genes across vertebrates; for example, of 1001 miR-181/a/b/c/d target genes predicted in mice, 914 are also predicted targets in humans (per TargetScan database). The miR-181a/b/c/d family is highly-enriched for expression in the nervous system with highest levels in post-mitotic neurons (including DA neurons), particularly for miR-181a/b12-14; one miR profiling study in mouse brains indicated that miR-181a is consistently expressed in the top 20 of over 600 detected miRs across several neuronal sub-types. Furthermore, we and others have shown that the miR-181 family targets several genes involved in neuronal growth and neurite extension, mitochondrial biogenesis, autophagy/mitophagy, and cell survival. Notably, genetic and molecular inhibition of miR-181a/b is neuroprotective in fish and rodent models of genetically- or chemically-induced mitochondrial dysfunction, which present with neurodegenerative phenotypes, as well as in mice subjected to cerebral ischemia (i.e., stroke). Based on these overall findings, we hypothesized that miR-181 inhibition may be neuroprotective in a PD mouse model, and we therefore tested the effects of viral-mediated gain- and loss-of-function of miR-181 in a viral-based PD model of aSyn overexpression. The results of our studies support that miR-181 promotes DA neuronal degeneration and suppressing its activity protects against aSyn-induced neurotoxicity.

### Results

### miR-181 Target mRNAs are Broadly Depressed in Aging and PD Brains

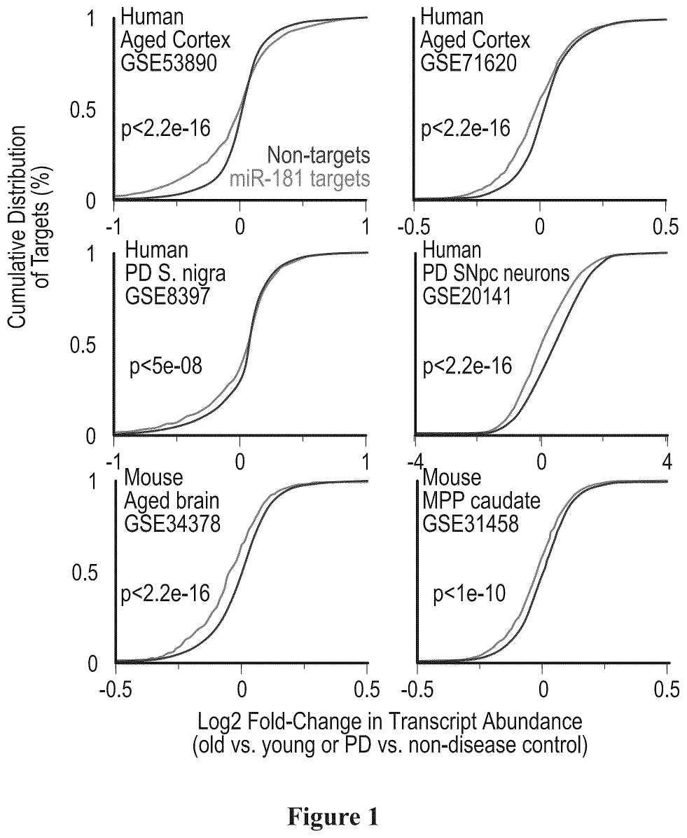

Previous reports indicate that miR-181a levels are elevated in aging and PD brain tissues; however, there have been no published efforts attempting to link these changes with broad down-regulation of miR-181 target genes in these settings. To address this, we analyzed publicly available data from several studies that previously evaluated transcriptomic changes in human brain samples, comparing young versus old, or control versus PD tissues. Specifically, we performed cumulative fraction analyses to compare fold-change distributions of miR-181 target genes versus all other genes. Notably, in each dataset, we found that miR-181 mRNA targets are overall significantly shifted towards lower expression levels in aged cortex (ages 24-29 versus 80+years-old, NCBI GEO Accession: GSE53890; and, ages 18-25 versus 65+years-old, GSE71620), as well as in SN tissues (GSE8397) and laser-microdissected DA neurons (GSE20141) from PD versus non-diseased control samples (FIG. 1).

### Generation of miR-181 Overexpression and Inhibition Vectors

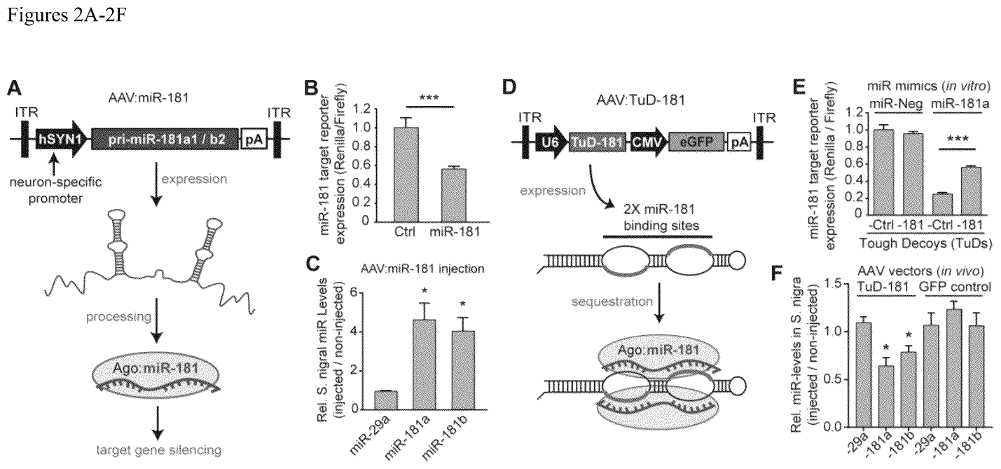

As an initial step towards testing the effects of gain- and loss-of-function of miR-181 on DA neurons in the setting of PD, we constructed plasmids and viral vectors to overexpress or inhibit miR-181. For overexpression, we generated a dual-hairpin miR-181a1/b2 primary miR transcript driven by either a CMV promoter for preliminary in vitro testing or a neuronal-specific human synapsin-1 gene promoter (hSYN1) for subsequent in vivo testing in mouse brain (FIG. 2A). In cell transfection studies, the CMV-based miR-181a1/b2 plasmid increased miR-181a/b levels by >30-fold (p<0.001, data not shown) and repressed the expression of a co-transfected luciferase reporter gene harboring miR-181 seed binding sites (˜50%, p<0.001; FIG. 2B). For in vivo testing, a neurotropic recombinant adeno-associated virus (AAV, serotype 5) encoding the hSYN1-miR-181a1/b2 cassette was delivered unilaterally to the SN in mice via direct intra-parenchymal injection. Three weeks later, this led to a >4-fold increase in miR-181a/b levels in injected SN, relative to non-injected contralateral side controls (p=0.001, FIG. 2C). While this experiment lacks a control AAV injection into the contralateral side, this increase is not likely the result of a general response to direct AAV injection, since miR-181a levels do not significantly increase following AAV-GFP injection (FIG. 2F).

For miR-181 inhibition, we generated a U6-driven “tough decoy” (TuD) RNA that forms a highly stable stem-loop structure capable of sequestering two miR-181 molecules via high-affinity antisense binding sites (FIG. 2D). In cell transfection studies, the TuD-181 plasmid construct reversed the silencing of a miR-181 target luciferase reporter by ˜50% (p<0.0001, relative to a scrambled control, TuD-6 Ctrl), in the presence of elevated miR-181 levels, achieved via co-transfection of synthetic miR-181 mimic oligonucleotides (FIG. 2E). For in vivo testing, an AAVS co-expressing U6-driven TuD-181 and CMV-driven GFP (AAV:TuD-181) was injected unilaterally into the SN in mice. Nine weeks later, endogenous miR-181a/b levels were reduced 20-35% in injected SN, relative to either non-injected contralateral side controls or control mice that received unilateral injection of AAVS expressing GFP alone (AAV:GFP; p=0.001, FIG. 2F), whereas expression of another abundant brain miR, miR-29a, was not changed. These findings are noteworthy and suggest that the magnitude of miR-181 inhibition may be greater than detected by this assay, considering that TuD RNAs act primarily through miR sequestration, as opposed to degradation.

### Generation and Characterization of an AAV-Based aSyn Overexpression Mouse Model of PD

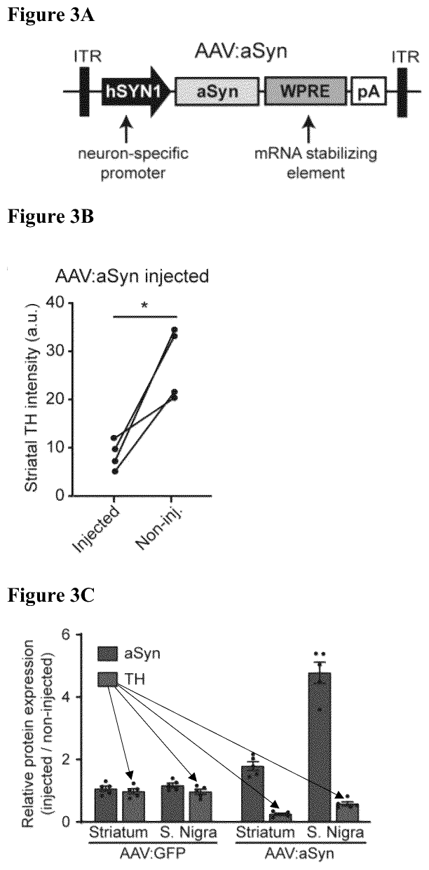

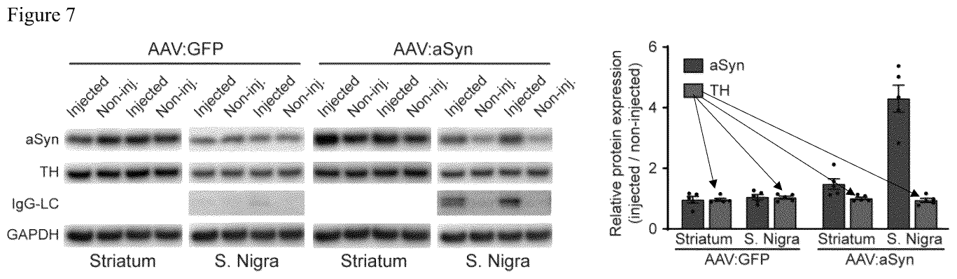

To test the effects of viral-mediated gain- and loss-of-function of miR-181 on PD outcomes in mice, we first established an AAV-based aSyn overexpression PD model to work in our hands. For this, AAVS encoding human aSyn driven by the neuron-specific hSYN1 promoter (AAV:aSyn) was unilaterally injected directly into the SN in mice (FIG. 3A). After 2-3 weeks post-injection, aSyn expressed from the AAVS transgene was evident in the SN by immunostaining using an antibody specific for human aSyn. Western blotting SN tissue lysates for total aSyn (human transgene and endogenous mouse) showed a 4-fold increase in aSyn levels, relative to either non-injected contralateral side controls or AAV:GFP-injected mice (p<0.0001, one-way ANOVA with Tukey multiple comparison's test; FIG. 7). At these early time-points, aSyn overexpression did not adversely affect DA neurons in the SN nor their projections to the striatum, as evidenced by preserved tyrosine hydroxylase (TH) immunostaining and western blotting (FIG. 7). However, at later time points (9-18 weeks), significant loss of TH staining was found in AAV:aSyn injected hemisphere in both the striatum (loss of DA neuronal projections) and SN (DA neuronal cell loss) and approached that obtained after unilateral injection 6-hydroxydopamine (6-OHDA, a classic nigral neurotoxin; FIGS. 3B). Reduced TH protein levels were substantiated by western blot, wherein TH bands were overtly diminished in SN and striatal regions of AAV:aSyn mice (injected vs non-injected hemisphere) but not AAV:GFP mice, at 9 weeks post-unilateral injection (FIGS. 3C). Confocal imaging revealed coincident loss of neuronal cell bodies and TH+neurons in the injected SN, supporting a model in which aSyn overexpression precipitates DA neuronal death. Concomitant with TH loss, striatal staining for dopamine transporter (DAT, a marker of DA neuron terminals) was reduced, while staining for DARPP-32 (a marker for medium spiny neurons) showed no difference in injected versus non-injected sides. These findings are consistent with selective loss of DA neurons and their projections, while striatal target neurons remain intact. The aSyn-induced neurotoxicity was also accompanied by neuroinflammation, a classic hallmark of PD and other neurodegenerative diseases, evidenced by increased staining for activated microglia (Iba-1 marker) and infiltration of endogenous mouse immunoglobulins (IgG), the latter likely resulting from blood-brain barrier disruption. Increased levels of IgG-light chains in AAV:aSyn-injected SN versus non-injected and AAV:GFP controls were also observed by western blot (FIG. 7). Notably, our AAV:GFP injections are well-tolerated and provide a key control for potential adverse effects related to general protein overexpression, since prior studies have observed neurotoxic effects caused by excessive AAV-mediated GFP expression in TH neurons. Altogether, these data indicate that our AAV:aSyn model induces a consistent, time-dependent degeneration of TH+neuronal projections and cell bodies and neuro-inflammation, providing a useful model to assess the impacts of miR-181 modulation on these PD outcomes.

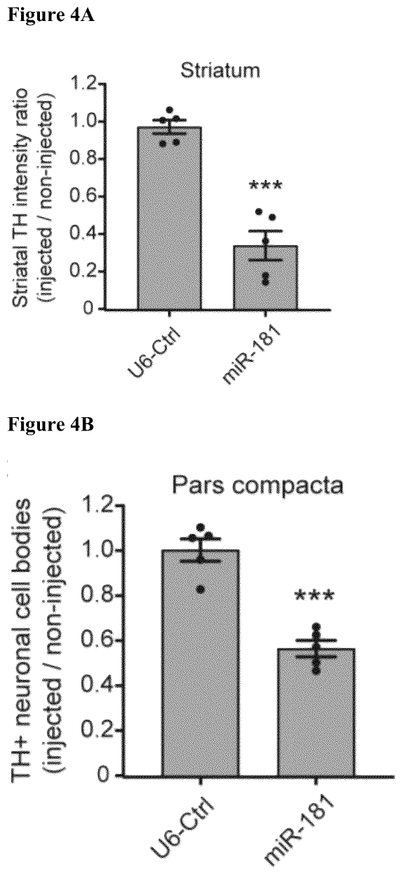

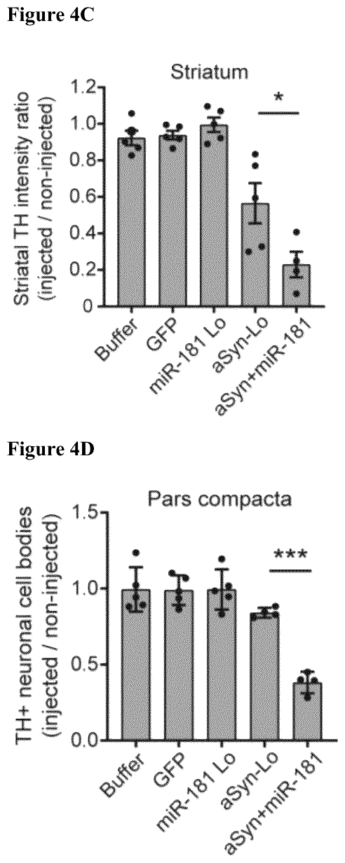

Effects of miR-181 overexpression on aSyn-induced neurotoxicity in mouse brain Prior independent reports indicate the miR-181a levels are increased in aged and PD human brain tissues, and our further interrogation of available datasets indicate that miR-181 target genes are coincidingly down-regulated (FIG. 1); however, the biological impacts of elevated miR-181 levels on DA neuronal health and aSyn-induced PD outcomes were unknown. To address this, we first evaluated the effects of elevated miR-181a/b in neurons in mice injected unilaterally with AAV:miR-181 (1.65e10 vg) into the SN. At 18 weeks post-injection, TH staining was reduced in AAV:miR-181-treated hemispheres, indicating neurotoxic effects of excess miR-181 alone. To verify the specificity of miR-181 neurotoxicity, additional mice were injected unilaterally with 1.65e10 vg of either AAV:miR-181 or AAV:U6-Ctrl, which expresses a scrambled miR control. After 9 weeks, AAV:miR-181 treatment led to —65% reduced striatal TH staining intensity (p<0.001) and ˜45% loss of SN dopaminergic neurons (p<0.001), relative to non-injected control sides; by contrast, TH staining was preserved in the AAV:U6-Ctrl group (FIGS. 4A-4B). Based on these observations, we hypothesized that elevated miR-181 may promote PD progression, and in the setting of aSyn overexpression, would cause enhanced neurotoxicity. To address this hypothesis, we performed co-injections of AAV:aSyn plus AAV:miR-181, with dosing adjusted to test miR-181-mediated exacerbation. We used a lower dose of AAV:aSyn to elicit intermediate TH loss (2.5e⁹ vg), and we also lowered the amount of miR-181 vector to a non-toxic dose (2.5e⁹ vg). At 16 weeks post-injection, AAV:aSyn alone caused moderate DA neuron loss, based on striatal TH staining intensity as well as nigral TH+neuronal counts (injected vs non-injected; FIGS. 4C-4D). In comparison, co-injection of AAV:aSyn and AAV:miR-181 elicited a striking exacerbation of TH loss; striatal TH staining was severely reduced (-80% decline, p<0.05) in injected hemispheres, and —60% of TH+neurons were lost in the pars compacta of the SN (p<0.0001). AAV:miR-181 alone at this same dose had no effect on either of these readouts, similar to buffer-injected and AAV:GFP-injected controls. Together, these findings support that miR-181 overexpression is sufficient to induce neurotoxicity in dopaminergic neurons and can synergize with aSyn to further provoke neurotoxic outcomes in PD.

### Effects of miR-181 Inhibition on aSyn-Induced Neurotoxicity in Mouse Brain

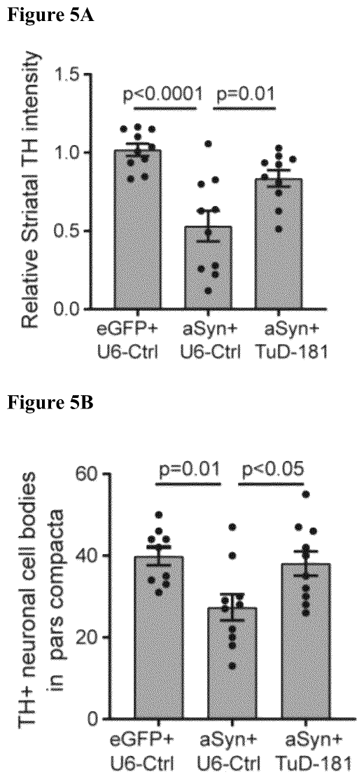

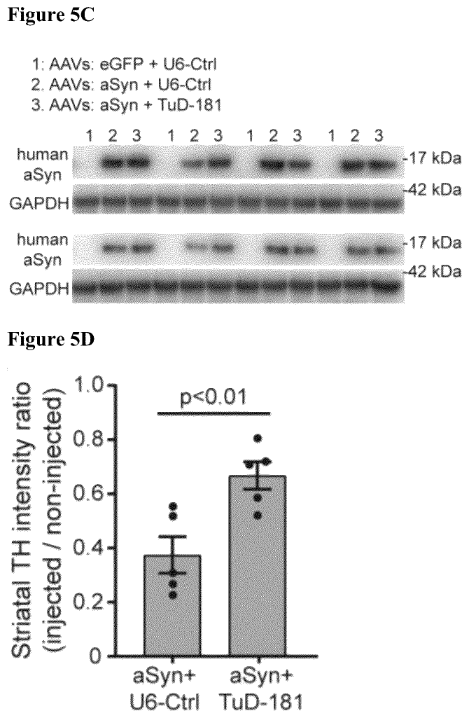

Considering that miR-181a is elevated in human PD brains and that increased miR-181 exacerbated aSyn-induced neurotoxicity in mouse brain, we hypothesized that blocking endogenous miR-181 may provide neuroprotection in the setting of aSyn overexpression. To address this, we first tested the effects of AAV:TuD-181 treatment alone following direct injection into mouse SN. At 18 weeks post-injection, AAV:TuD-181-injected (2e10 vg) and non-injected hemispheres were indistinguishable by TH staining, supporting that prolonged TuD-181 RNA expression and miR-181 inhibition is well-tolerated in DA neurons. We next conducted a co-injection study with AAV:aSyn (1.3e10 vg) plus either AAV:TuD-181 or AAV:U6-Ctrl (1e10 vg). In addition, AAV:GFP (1.3e10 vg) plus AAV:U6-Ctrl (1e10 vg) served as control for potential artifacts related to protein and non-coding RNA overexpression. All injections were done bilaterally into the SN to allow assessment of motor deficits. Accelerated rotarod and open-field ambulatory measures were collected from 15 to 20 weeks post-injection. However, AAV:aSyn⁺AAV:U6-Ctrl mice did not exhibit any robust and reproducible deficits in motor behavior compared to the control groups (AAV:aGFP⁺AAV:U6-Ctrl or non-injected mice, data not shown), precluding the ability to determine significant influence of miR-181 manipulation on motor phenotypes in this model. Histological analyses performed at 24 weeks post-injection showed that mice treated with AAVs:aSyn⁺U6-Ctrl exhibited a marked reduction in striatal TH staining intensity relative to AAV:GFP control injected mice (˜50% decline, p<0.0001; FIG. 5A), consistent with our previous observations with unilateral injections of AAV:aSyn (FIGS. 3B). AAVs:aSyn⁺U6-Ctrl injection also resulted in significant loss of TH+neuronal cell bodies in the SN pars compacta (SNpc; ˜30% decline, p=0.01; FIG. 5B). By contrast, mice injected with AAVs:aSyn⁺TuD-181 showed a remarkable protection against aSyn-induced neurotoxicity, as evidenced by minimal (˜15%) loss of striatal TH staining and clear preservation in the numbers of TH+neurons in the SNpc (only ˜5% decline). Relative to control AAVs:aSyn⁺U6-Ctrl mice, this protection was significant (p=0.01 and p<0.05 for striatal and SN TH+staining respectively, FIGS. 5A-5B). This protection was not associated with lower aSyn transgene expression, as levels of human aSyn were similar in SN regions of AAVs:aSyn⁺U6-Ctrl and AAVs:aSyn⁺TuD-181 injected mice (FIG. 5C). To assess the reproducibility of this TuD-181-mediated protective effect, we performed unilateral injections of either AAVs:aSyn⁺U6-Ctrl or AAVs:aSyn⁺TuD-181 and determined the striatal TH staining intensities (injected relative to non-injected hemisphere) 16 weeks later. Consistent with our prior results, TH loss was significantly less severe in the AAVs:aSyn⁺TuD-181 treatment group compared to the AAVs:aSyn⁺U6-Ctrl injected group (p<0.01; FIG. 5D), further supporting that blockade of endogenous miR-181 protects against aSyn-induced neurotoxicity.

### MiR-181 Suppresses a Diverse set of PD-Related Gene Targets

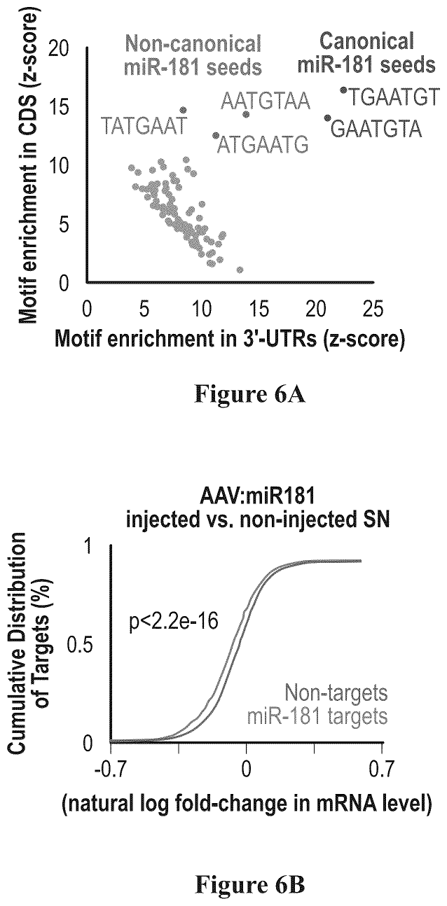

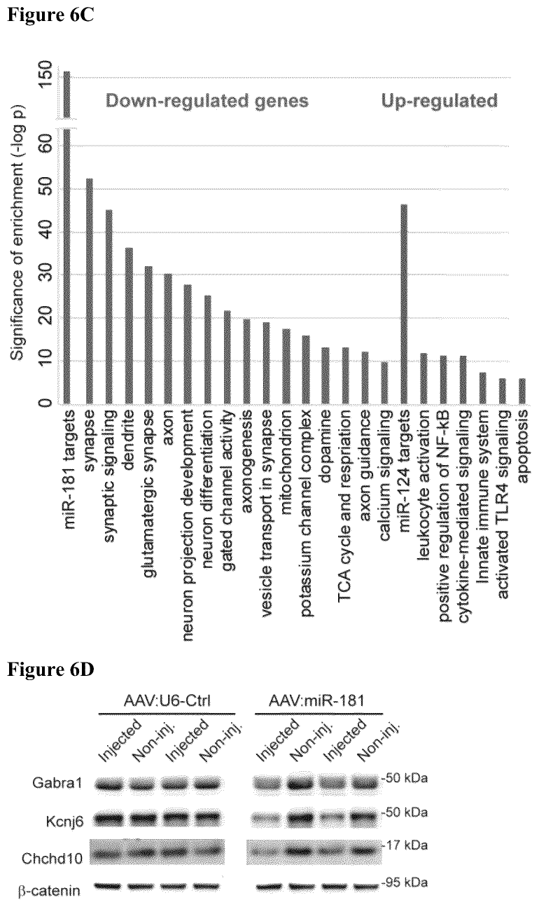

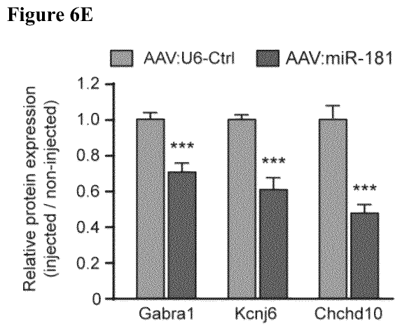

To gain mechanistic insight into how miR-181 may influence neuronal function and viability in the SN, we performed mRNA-sequencing on SN RNA samples collected from mice injected unilaterally with AAV:miR-181 to evaluate gene expression changes relative to non-injected contralateral control sides, while comparing the resulting data to mice injected unilaterally with AAV:GFP control virus to account for changes simply resulting from direct AAV injection. Differential expression analyses were done yielding many significantly altered genes across treatment groups, with an obvious broad down-regulation of miR-181 targets in mice injected with AAV:miR-181, which was not observed with AAV:GFP injection. Also, many significantly up-regulated genes were found in injected vs. non-injected hemispheres, likely due to an inflammatory response related to direct AAV injection; notably, this was more pronounced in AAV:miR-181 versus AAV:GFP samples. Unbiased query of heptamer motif frequencies in mRNAs revealed that heptamers corresponding to miR-181 seed sequences were the most significantly enriched heptamers among down-regulated mRNAs (across both coding and 3′-untranslated regions, 3′-UTRs) in miR-181 overexpression mice (FIG. 6A). In addition, cumulative distribution analyses confirmed that miR-181 mRNA targets are overall significantly shifted towards lower expression levels (FIG. 6B). Altogether, these findings provide strong support that our neuron-targeted AAV-mediated miR-181 overexpression strategy robustly induces the expected down-regulation of a diverse set of miR-181 target mRNAs. Gene set enrichment analyses [using ToppFun, ToppGene Suite] performed on genes that were significantly down-regulated in AAV:miR-181 samples (p<0.05), but not in AAV:GFP samples (p>0.2), indicated that miR-181 overexpression broadly coordinated the repressions of genes involved in synaptic signaling, neurite and axonal projection, ion transport, mitochondrial metabolism/respiration, and calcium signaling (FIG. 6C).

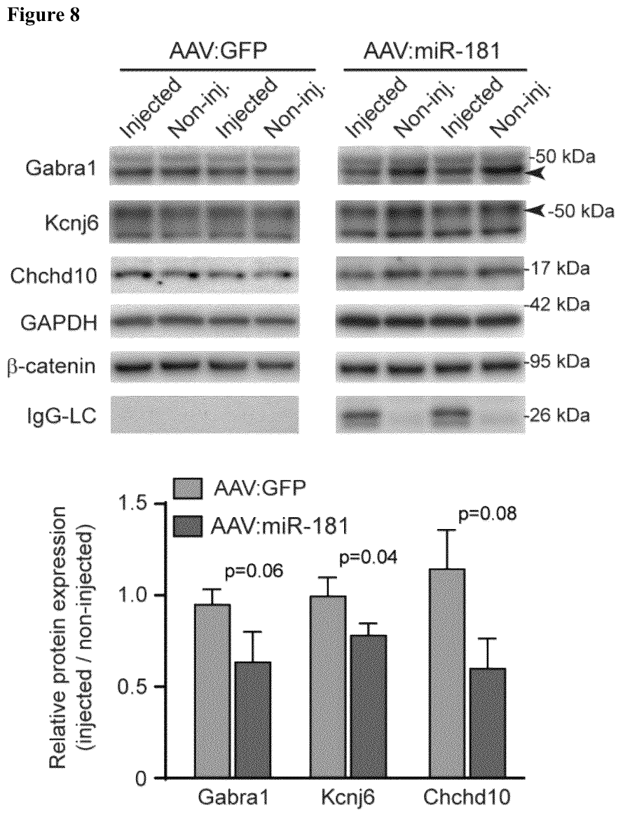

Beyond the down-regulated genes, we found that genes showing significant increases in expression following AAV:miR-181 treatment (p<0.05), but not increased with AAV:GFP (p>0.2), were enriched for immune system pathways (e.g., leukocyte activation and positive regulation of NF-kB, cytokine, and TLR4 signaling), suggesting that the neuronal overexpression of miR-181 may trigger neuroinflammatory responses above and beyond that caused merely by direct AAV injection. These findings were further supported in subsequent western blot analyses that revealed an abundance of mouse IgG-light chains in AAV:miR-181 treated SN lysates (FIG. 8), similar to that observed with AAV:aSyn treatment (FIG. 7); by contrast, IgG-light chains were only weakly detected in AAV:GFP injected brains (FIG. 8).

We next chose select targets from our mRNA-seq results to validate miR-181-mediated target suppression at the protein level. We focused on targets with miR-181 binding sites (computationally-predicted and/or miR-181 sites physically bound by Ago2-RISC complexes in human and mouse tissues, including brain, and further narrowed selection based on: i) expression in SN neurons, ii) relevance to DA neuronal function, and iii) availability of antibodies. Three genes that met these criteria were Gabral, Kcnj6, and Chchd10, and these represent various types of miR-181 binding sites (e.g., a canonical Gabra1 3′UTR site found by TargetScan prediction and Ago2-pulldown, a Kcnj6 3′UTR site missed by TargetScan due to poor 3′UTR annotation, but found by Ago2-pulldown, and a non-canonical Chchd10 3′UTR found by Ago2-pulldown. Gabra1 encodes the alpha 1 subunit of GABAA inhibitory neurotransmitter receptors, which are critical to the basal ganglia-SN circuitry regulating dopamine-driven movement. Kcnj6 encodes Girk2, a G-protein activated potassium channel that provides negative feedback to limit neuronal firing and dopamine release. Chchd10 encodes a coiled-coil-helix-coiled-coil-helix domain containing protein that partners with Chchd2 to support mitochondrial cristae maintenance and respiratory function. Notably, mutations in Chchd10 and Chchd2 have been linked to frontotemporal dementia and ALS and PD, respectively. Initial western blotting of protein lysates collected from mice used for the RNA-seq study showed trends toward reduced levels of Gabral, Kcnj6 and Chchd10 proteins, respectively, in AAV-miR-181-injected SN relative to non-injected SN (p<0.1), but no significant change after AAV-GFP injection (n=3-5/group; FIG. 8). To increase sample numbers and include a more appropriate control group, additional mice were injected unilaterally with 1.65e10 vg of either AAV:miR-181 or AAV:U6-Ctrl, which expresses a scrambled miR control. Western blotting of SN samples collected from these mice at 3 weeks post-injection showed significantly decreased (30-50%) Gabral, Kcnj6 and Chchdl0 protein levels in AAV-miR-181-injected SN relative to non-injected control sides (p<0.001), while AAV:U6-Ctrl mice showed no unilateral differences in expression of these proteins (FIGS. 6D, 6E). Together, these data demonstrate that miR-181 functionally suppresses mRNA levels for hundreds of gene targets with diverse functions and potential relevance to PD pathogenesis and provide general support that the observed mRNA decreases are also reflected at the protein level.

### Discussion

This study began with an interest in the potential relevance of increased miR-181 levels in PD patient samples. We now show that elevated miR-181 coincides with broad decreases in miR-181 target gene mRNAs in both aged and PD brain samples collected from human patients and rodent models. Our analyses indicate a trend towards larger decreases in miR-181 target gene mRNAs in aged versus young compared to PD versus control samples, suggesting that natural aging more profoundly enhances miR-181 functions and that this may contribute to provoking PD onset and progression with advanced age. One important limitation worth noting is that these public data lack information on actual miR-181 levels in each of the samples, and thus these analyses are only capable of correlatively, not directly, linking the prior separately published observations of miR-181 elevations in PD and aging brains to broad decreases in miR-181 target gene expressions, as we show in FIG. 1. To understand whether miR-181 augments or antagonizes PD, we manipulated miR-181 levels in the context of a PD mouse model, elicited by AAV5 vector mediated overexpression of human aSyn in the SN. Collectively, our findings indicate that elevated miR-181 exacerbates, while blockade of endogenous miR-181 diminishes neuronal loss incurred by aSyn overexpression.

For our AAV-based PD model, we chose the serotype 5 capsid, previously demonstrated to transduce mouse DA neurons, with the aSyn transgene cassette incorporating the neuronal specific synapsin-I promoter plus WPRE elements, shown by others to enhance SNpc loss and disease severity. At three weeks post-injection into the SN (1e10 vg dose), we achieved an approximate 4-fold overexpression of aSyn, which triggered pathogenic events leading to significant loss of TH staining by 9 weeks. Our model is consistent with similar AAV generated rodent models, displaying salient features of PD including neuroinflammatory responses (IgG influx and reactive Iba+microglia), enhanced phosphorylated alpha synuclein (P-S129), and progressive loss of TH positive striatal projections and DA SNc neurons. Though we were unable to reliably detect significant deficits in rotarod performance or open field ambulatory parameters, this was not surprising since PD-related motor dysfunction typically does not manifest unless SNpc neuron loss exceeds approximately 60%, a level that was not consistently reached in our studies. Nevertheless, our model displayed characteristic pathological PD features, with significant loss of TH+projections/neurons, indicative of SNpc DA dysfunction, degeneration and death, providing indicators of disease severity to gauge the effects of miR-181 modulation.

Interestingly, we found that ˜4-fold overexpression of miR-181a/b alone was toxic to DA neurons, which is compatible with reports implicating miR-181 in provoking cell death in stroke, seizure or mitochondrial disease models. When delivered at a lower non-toxic dose, and co-injected with AAV:aSyn, AAV:miR-181 markedly exacerbated the extent of aSyn-induced striatal TH loss, worsening it from ˜45% to —80%. This leads us to speculate that aging-related increases in miR-181 levels could hasten the onset and progression of PD in patients. Conversely, we found that co-injection of AAV:TuD-181 with AAV:aSyn had the opposite effect, protecting against aSyn-induced neuronal toxicity (50-65% vs 15-30% striatal TH loss), supporting the need to further explore the therapeutic potential of miR-181 inhibition in the setting of PD. Beyond our gene therapy approach, this is supported by additional studies which demonstrated neuroprotection in mice with genetic reduction of miR-181 expression, achieved by partial knockout strategies.

Despite several studies supporting that miR-181 may promote neuronal dysfunction and death, the mechanisms by which this occurs remain unclear due to the lack of comprehensive empirical data for miR-181 functional targeting. Here, we describe the first transcriptome-wide gene expression dataset characterizing the effects of miR-181 overexpression in rodent brain. This dataset reveals that miR-181 operates by suppressing a diverse set of gene targets, 282 of which harbor cross-species conserved miR-181 sites predicted by TargetScan. While others have attributed the neuroprotective effects of miR-181 inhibition to its regulation of genes that block apoptosis or influence mitochondrial function and biogenesis, our dataset points to an even broader effect, with many miR-181 gene targets operative in synaptic signaling, ion transport, calcium signaling, and mitochondrial function, all of which likely contribute to shaping the course of PD. For example, several notable down-regulated miR-181 targets include genes that are protective in PD model systems (e.g., Kcnh1, Slc2a3, Atg5, and Nrg1) or play important roles in establishing or maintaining the DA neuronal cell phenotype (e.g., Rspo2 and Bmp2). Additionally, our RNA-seq data support that AAV:miR-181 injection, relative to AAV:GFP controls, results in enhanced immune activation and increased expression of miR-124 target genes, hinting at possible miR-181-mediated miR-124 inhibition by unknown mechanisms. The latter is noteworthy since prior reports indicate that miR-124 protects against neurodegeneration, including studies in PD models.

Given that miRs are known to coordinate biological responses through targeting multiple genes within a shared pathway, it is conceivable that in a disease setting, miR-181 regulates a defined subset of genes that sway neurons to their demise. Such a gene set may be unique to PD or extend to other synucleinopathies (e.g., dementia with Lewy bodies) or even more broadly to neurodegenerative disease/neuropathy. While our mRNA-seq data provide a great resource to better understand miR-181 functions in brain, it represents a snapshot of gene expression at three weeks post miR-181 over-expression. To better discern potential mechanisms related to therapeutic manipulation of miR-181, a more comprehensive mRNA-seq study is needed to further define miR-181 target gene fluctuations in the setting of and through the course of PD. Nevertheless, the current dataset can be further queried for additional miR-181 target interactions related to PD and beyond. For example, miR-181 has been implicated in stroke-induced neuronal death and was found to be up-regulated in response to cocaine, having potential involvement in cocaine addiction. Notably, our gene enrichment analyses (via ToppFun) identified cocaine as the top hit the drug category, indicating an alignment of cocaine-related genes and our miR-181 down-regulated gene set in mouse brain.

In summary, our new data are compelling and suggest that miR-181 up-regulation provokes DA neuronal cell death and accelerates aSyn-induced neurotoxicity. It is interesting to note that several pesticides previously linked to risk of PD manifestation have been shown to elicit robust increases (˜3-4-fold) in miR-181 levels in various experimental settings.

### Materials and Methods

Plasmids. To create the “miR-181 target reporter” plasmid, a synthetic DNA oligo with the sequence:

(SEQ ID NO: 5)
5′-CTCGAGGTGAATGTTACCTTGAAATGCTCCAACCTGAA
TGTTAGGTTTGCCGTTGAATGTTAGCGGCCGC-3′

containing three miR-181 binding sites (IDT, Coralville, IA) was introduced downstream of the Renilla luciferase expression cassette (using XhoI and NotI restriction enzyme sites) in the psiCheck2 dual luciferase plasmid (Promega, Madison, WI). pAAV2/5-U6:TuD-Ctrl (TuD-Ctrl) and pAAV2/5-U6:TuD-181 (TuD-181) shuttle plasmids were constructed by inserting DNA oligo sequences (IDT) to express the following TuD stem-loop sequences from the U6 promoter:

(SEQ ID NO: 6)
GACGGCCTCGAGACTGGAACCTACACAATGCAGAAATACCACAA
GTATTCTGGTCACAGAATACAACCATCACAATGCAGAATAACCA
CAACTAGTCTCGGGGCCGTCTTT

### for TuD-Ctrl, and

(SEQ ID NO: 7)
GACGGCCTCGAGACTGGCAACTCACCGACAAAGATGAATGTTCA
AGTATTCTGGTCACAGAATACAACCTTACCGACAAAGATGAATG
TTAACACTAGTCTCGGGGCCGTCTT

for TuD-181, both cloned into a custom version of pFBAAVmU6mcsCMVeGFPSV40pA (ID #G0347 University of Iowa Viral Vector Core Facility, Iowa City, IA; UIOWA VVC). CMVmiR-181a1b2 plasmid was constructed by ligating a custom gBLOCK DNA fragment containing tandem miR-181a1 and miR-181b2 sequences (˜450-500bp of human genomic sequence centered on each miR loop) into pFBAAVCMVmcspBgHpA (ID# G0347 UIOWA VVC). CMV-SNCA plasmid was created by blunt ligation of a synthetic DNA gBLOCK (IDT) of human SNCA cDNA with flanking restrictions sites (SpeI-BglII-SNCA-MfeI-SpeI) into pCR®-Blunt-II-TOPO (Thermo Fisher Scientific, Waltham, MA) followed by digest of TOPO-SNCA with SpeI and ligation into NheI-digested pFBAAVCMVmcspBgHpA (ID# G0347 UIOWA VVC). To create the pAAV2/5-hSYN1-SNCA shuttle plasmid, an AAV shuttle plasmid with the human synapsin-1 promoter and a WPRE (Plasmid #50465 Addgene, Watertown, MA) was digested with BamHI and EcoRI, dropping out the eGFP. The SpeI-BglII-SNCA-MfeI-SpeI gBLOCK was digested with BglII and MfeI and ligated into the AAV shuttle, replacing the eGFP. To create the pAAV2/5-hSYN1-miR-181a1b2 shuttle plasmid, standard cloning techniques were used to remove eGFP from the parental shuttle plasmid (Plasmid #50465 Addgene) and replace it with the gBLOCK DNA fragment containing tandem miR-181a1 and miR-181b2 sequences.

AAV2/5 vectors. All AAV vectors were prepared at the UIOWA VVC and contain vector genomes flanked by AAV2-type ITRs, packaged into AAV serotype 5 capsids, by standard triple transfection (3XT) or baculovirus (BAC) methods. AAV2/5hSynEGFP (AAV:GFP; 3XT) and AAV2/5mU6-miSafeCMVeGFP (AAV:U6-Ctrl; BAC) were purchased as in-stock vectors from UIOWA VVC (catalog #s VVC-U of Iowa-3412 and VVC-U of Iowa-258). Shuttle plasmids pAAV2/5-U6:TuD-181, pAAV2/5-hSYN1-miR-181a1b2, and pAAV2/5-hSYN1-SNCA described above were supplied to the UIOWA VVC to generate custom AAV preparations of AAV:TuD-181 (BAC), AAV:miR-181 (3XT) and AAV:aSyn (3X), respectively. AAV purification was done with an iodixanol gradient followed by ion exchange using MustangQ Acrodisc membranes (Pall, East Hills, NY), and titers of purified AAV preparations (vector genomes per ml, vg/ml) were determined by QPCR.

Test of TuD181 construct in N2A cells. Mouse Neuro2a (N2a) cells (CCL-131, ATCC, Manassas, VA) were seeded at 80,000 cells/well in 24-well tissue culture dishes and the next day cells were co-transfected with 100 ng miR-181 target reporter plasmid plus 4 nM either control or miR-181 pre-miR synthetic oligos (Ambion, Austin, TX), along with 100 ng either TuD-Ctrl plasmid or TuD-181 expression plasmid, using Lipofectamine 2000 (Thermo Fisher Scientific). Each combination was assayed in triplicate culture wells. At 48 h post transfection Firefly (FF) and Renilla (R) luciferase activities were measured using a GloMax Microplate Reader and Dual Luciferase Kit reagents (Promega). Briefly, culture media was removed and 200 ul Passive Lysis Buffer was added to each well, and the plate incubated on a shaker for 15 min at RT, then 10-μl lysate was transferred to duplicate wells of a 96-well white plate. Luminescence from FF and R was determined from 5 second reads after injection of respective substrates to each well. The R/FF ratio was calculated and adjusted relative to the control (set to “1”), and results are expressed as the mean ±SEM (n=4/group).

Test of CMVmiR-181a1b2 construct in HEK293. HEK293 cells (CRL-1573 ATCC) were seeded at 100,000 cells/well in 24-well plates and transfected 24 h later for use in either luciferase assay or RT-qPCR. For luciferase assay, cells were co-transfected with 20 ng of the miR-181 target reporter plasmid and 200 ng of either control parental plasmid (CMV-only), CMVmiR-181a1b2, using Lipofectamine 2000. At 48 h post transfection, lysates were collected from cultures and transferred to duplicate wells for luminescence readings. Luminescence from FF and R was determined from 5 second reads after injection of respective substrates. The R/FF ratio was calculated and adjusted relative to the control (set to “1”), and results are expressed as the mean ±SEM (n=3 for CMV-only control; n=6 for CMVmiR-181a1b2.

Stereotaxic injections. Experiments with mice were performed in accordance with University of Iowa Animal Care and Use Committee (IACUC) regulations. C57Bl/6J mice were purchased from JAX (stock# 000664, JAX, Bar Harbor, ME) and housed under 12/12 h light/dark cycle with access to food and water ad libitum. Stereotaxic injections were performed on female C57BL/6J mice at 8 to 11 weeks of age. Under anesthesia, the head was shaved and treated with antiseptic and the mouse placed in a stereotactic head frame (Stoelting, IL, USA). A midline incision was made in the scalp and a small burr hole was drilled at appropriate coordinates. Using a 33-gauge blunt needle on a 5-ul Neuros Syringe (Hamilton Company, Reno, NV), 1.5 μl of vector was injected at a rate of 0.2 μl min, at coordinates anteroposterior (AP) −3.2 mm, mediolateral (ML) 1.3 mm, and dorsoventral (DV) −4.2 mm from dura to target the SN region. The needle was left in place for an additional 5 min before slowly withdrawing over 2 min. For 6-hydroxydopamine (6-OHDA) injections, 6-OHDA (6-hydroxydopamine hydrobromide, Sigma-Aldrich, St. Louis, MO) was dissolved at 1 mg/ml (free base concentration) in in 0.9% saline with 0.03% acetic acid, made fresh before use. Using stereotaxic injection procedures as described above, 1 μl was injected in the medial forebrain bundle at coordinates AP −1.2 mm, ML −1.2 mm, and DV −4.75 mm from dura. Control mice received 0.03% acetic acid in 0.9% saline injection. 6-OHDA-injected and control mice received pre-surgery i.p. injection of 25 mg/kg desipramine HCl (Sigma-Aldrich), dissolved in saline, to protect noradrenergic neurons. All mice were given 1.0 ml lactated Ringers s.c. to provide hydration during surgical recovery.

Antibodies and stains. Antibody and dilutions were as follows: tyrosine hydroxylase (AB152 rabbit polyclonal IgG, MilliporeSigma, Burlington, MA) 1/1000 for staining tissue sections and 1/3500 for western blot; alpha synuclein (sc-58480 mouse IgG1 clone LB509, specific for human, SCBT, Dallas, TX) 1/500 for staining tissue sections and 1/1000 for western blot; phospho 5129 alpha synuclein (ab51253 rabbit monoclonal IgG, Abcam, Cambridge, MA) 1/1000 for staining tissue sections and 1/3000 for western blot; alpha-synuclein (BD 610786 mouse IgG, clone 42, reacts with human and mouse, BD Biosciences, San Jose, CA) 1/1000 (0.25 μg/m1 final) for western blot; GAPDH (AC002 mouse IgG, Abclonal, Woburn, MA) 1/30000 for western blot; beta catenin (PLA0230 rabbit polyconal, Sigma-Aldrich) at 1/3000 for western blot; Iba1 (019-19741 rabbit polyclonal, Fujifilm Wako Chemicals, Richmond, VA) 1/5000 for staining tissue sections; dopamine transporter (MAB369 rat monoclonal IgG clone DAT-Nt, Sigma-Aldrich) at 1/2000 for staining tissue sections; Darpp-32 (MAB4230 rat monoclonal IgG clone #375604, R&D Systems, Minneapolis, MN) at 1/500 (0.5 μg/m1) for staining tissue sections; GFP (A-6455 rabbit IgG, Thermo Fisher Scientific) 1/2500 for tissue staining; beta-actin (A5441 mouse mAb IgG clone AC-15 ascites, Sigma-Aldrich) 1/20000 for western blot; CHCHD10 (25671-1-AP, rabbit polyclonal antibody, Proteintech, Rosemont, IL) at 1/750 (0.4 μg/ml) for western blot; GABRA1 (12410-1-AP, rabbit polyclonal antibody, Proteintech) at 1/2000 (0.1 μg/ml) for western blot; GIRK2/Kcnj6 (21647-1-AP, rabbit polyclonal antibody, Proteintech) at 1/1000 (0.4 μg/ml) for western blot; HRP-conjugated goat anti-mouse IgG (115-035-146 Jackson ImmunoResearch, West Grove, PA) 1/2000 for staining tissue sections and 1/20000 for western blot; HRP-conjugated goat anti-rabbit IgG (111-035-144 Jackson ImmunoResearch) 1/2000 for staining tissue sections and 1/80000 for western blot; HRP-conjugated goat anti-mouse IgG light chain (115-035-174 Jackson ImmunoResearch, West Grove, PA) 1/20000 for western blot; HRP-conjugated goat anti-rat IgG (112-035-167 Jackson ImmunoResearch) 1/2000 for staining tissue sections; Alexa Fluor conjugated goat anti-rabbit or goat anti-mouse IgG (Thermo Fisher Scientific) 1/2000 for staining tissue sections. AlexaFluor488 conjugated wheat germ agglutinin (A488WGA) and ToPro 3 (Thermo Fisher Scientific) were used at 1/1000 for tissue section staining.

Tissue processing for RNA/protein analyses. For collection of SN and striatal regions for RNA and/or protein analyses, brains were harvested from mice after isoflurane overdose and cervical dislocation and immediately submerged for 5 min in ice-cold PBS before collecting slices. The coronal brain matrix (Precision Brain Slicer, Braintree Scientific, Inc., Braintree, MA) and all instruments and single-edge razor blades were pre-cooled and kept on ice. Slices were collected caudal to rostral, with the first blade inserted just rostral to the cerebellum. After placement of the second blade in the next slot, the first blade, carrying a 1-mm coronal brain slice, was removed and placed tissue-side up on an ice-cold block. This procedure of “next” blade placement and previous blade retrieval was continued rostrally through the brain. Regions of SN and striatum were dissected from the slices, snap-frozen in liquid nitrogen and stored at −80 until processing. Tissue pieces were homogenized in 150-200 ul ice-cold lysis buffer (50 mM Tris, 120 mM NaCl, 5 mM EDTA, 1% triton-X100) with protease and phosphatase inhibitors (Sigma-Aldrich or Thermo Fisher Scientific) using disposable pellet pestles. For RNA isolation, approximately 75-100 μl of lysate was transferred to 1-ml of TRIzol (Thermo Fisher Scientific) and stored at −80° C. until processing. To the remaining lysate, SDS and sodium deoxycholate detergents were added to final 0.1 and 1%, respectively, and lysates were sonicated with a probe sonicator (two rounds of 3 s, at a setting of 3) to reduce viscosity. Lysates were clarified by 10-min 4° C. centrifugation at 10,000×g, and supernatants transferred to fresh tubes. Protein concentrations were determined by BCA (Thermo Fisher Scientific) and equilibrated for all samples. Samples were resolved by denaturing electrophoresis on precast 4-12% bis-tris gradient polyacrylamide gels using NuPage reagents (Thermo Fisher Scientific) and transferred to PVDF using NuPage transfer buffer with 10% ethanol and 0.01% SDS. After transfer, the lower region of the PVDF membrane (for detection of alpha synuclein) was fixed in 0.8% paraformaldehyde in PBS for 30 min. All membranes were incubated for 1 h in a block solution of 5% dry milk dissolved in tris-buffered saline with 0.1% tween-20 (TBST), followed by primary antibody diluted in 2.5% dry milk in TBST overnight at 4° C. Blots were incubated for 1 h with HRP-conjugated secondary and developed by chemiluminescence on a VersaDoc Imaging system with Quantity-One software (Bio-Rad, Hercules, CA). Band intensities were normalized to a loading control band (beta actin, GAPDH, or beta-catenin,) in the same lane.

Test of SNCA construct for protein. Our sequence for human SNCA cDNA was tested in vitro for protein expression using the CMV-SNCA expression plasmid. HT1080 cells (CCL-121, ATCC) were seeded at 50,000 cells/well in 24-well plates and transfected 24 h later with 300 ng CMV-SNCA (in triplicate culture wells) using Lipofectamine 2000. At 48 h post transfection, lysates were collected into 1% SDS with protease inhibitors (Roche), sonicated, and protein concentrations equilibrated. Samples were resolved by denaturing electrophoresis on precast polyacrylamide gels and western blot performed as described above, for detection of α-synuclein (human plus mouse) and beta-actin as loading control.

RT-qPCR. Total RNA was isolated from TRIzol according to the manufacturer's instructions (Thermo Fisher Scientific) and dissolved in TE (IDT) and quantified by nano-drop. RNA was DNase treated using DNA-free kit (Thermo Fisher Scientific). Reverse transcription (RT) and qPCR reactions for miR were performed essentially according to “TacMan Small RNA Assays” protocol using TaqMan MicroRNA Assays for two-step RT-qPCR (hsa-miR-181a ID#000480, hsa-miR-181b ID#001098, hsa-miR-29a ID#000412, snoRNA135 ID#001230) (Applied Biosystems, Thermo Fisher Scientific). Briefly, 15-ul RT reactions were set up containing 1× MultiScribe RT buffer, 2 mM dNTPs (0.5 mM each), 50 Units Multiscribe, 3.8 Units RNase Inhibitor, 24 ng of total RNA, and lx concentrations of each TacMan assay RT primer in the pool, on ice. Pools for RT primers were “miR-181a+miR-29a+snoRNA135” and “miR-181b+miR-29a+snoRNA135”. Thermocycler conditions for RT were 16° C. for 30 min, 42° C. for 30 min, 85° C. for 5 min and 4° C. hold. After RT, cDNA samples were diluted 5-fold by the addition of nuclease-free water. For qPCR, 10-ul reactions were set up in TaqMan® Universal Master Mix II with 4 ul diluted cDNA and lx concentration of appropriate TaqMan miR primer/probe set, in a 384-well plate in triplicate. qPCR reactions were performed on a ViiA™ 7 Real-time PCR system with QuantStudio™ software (Thermo Fisher Scientific). Relative quantification (ΔΔCt) was used to analyze results, using SnoRNA135 as the reference gene (endogenous control). For samples from unilateral injections, the results are expressed as the injected side relative to the non-injected side.

Immunostaining and image analyses. For brain histology mice were perfused with 10 ml ice-cold PBS followed by 15 ml ice-cold 4% PFA in PBS, brains removed, post-fixed 24 h at 4° C., then transferred to 30% sucrose in PBS at 4° C. After 2-3 days completion of sinking in sucrose, brains were covered in Tissue-Tek® O.C.T. Compound (EMS, Hatfield, PA) in Peel-a-Way embedding molds and frozen by setting molds in a dry ice/ethanol bath. Coronal 40-μm floating sections were cut on a cryostat and stored at −20° C. in anti-freeze medium (300 g sucrose, 300 ml ethylene glycol, to 1 L final volume with 0.1 M phosphate buffer pH 7.4). Prior to staining, sections were washed for 10 min in PBS and endogenous peroxidase activity was quenched by 20 min incubation in 1.5% H2O2 in PBS. Sections were washed in PBS then blocked/permeabilized by incubating for 2 h at RT in PBS containing 10% normal goat serum (NGS) and 0.3% triton-X100, then incubated overnight at RT with primary antibody diluted in PBS containing 0.1% triton-x100 and 1 NGS (Ab diluent). Sections were washed three times at 15 min each in PBS before incubating with HRP-conjugated secondary antibody at 0.5-1 μg/ml in Ab diluent for 2 h at RT. Sections were washed twice in PBS, and once in imidizole/acetate buffer (IAB) (10 mM imidazole, 83 mM sodium acetate, pH adjusted to 7.4 with glacial acetic acid), before developing in 3,4-diaminobenzoic acid (DAB) reagent (0.5 mg/ml DAB and 0.006% H₂O₂ in TAB) made fresh from stocks just prior to use. Care was taken to equilibrate development times for sections across groups within same experiments. Development was stopped by replacement of DAB reagent with TAB, followed by transfer to PBS. Sections were floated in 20 mM Tris pH 7.5 and mounted onto glass plus slides, dried overnight on the bench, then dehydrated through sequential 5 min incubations in 95%/100%/100% ethanol/zylene/zylene, then cover-slipped with Tek-Select® Clear Permaslip mounting medium (IMEB, San Marcos, CA). Bright-field images were captured on a BX53 upright scope with a DP72 camera and cellSens software (Olympus, Center Valley, PA). Capture settings were identical across slides within the same experiment. DAB staining intensities for TH stained striatum were determined using ImageJ/Fiji. Briefly, the striatal region (caudate putamen) was outlined using the drawing tool and the mean intensity (range of 1-255) was measured and inverted (255-mean intensity). Background (from a non-stained region of the cortex) was subtracted. For bilateral experiments, intensities for each striatum are expressed relative to the non-injected control group (set to “1”). For unilateral experiments, the intensity of the injected-side stratum is expressed relative to the non-injected side (set to “1”). Counts of TH+neurons in SNpc were done in blinded fashion using matched sections (rostra-to-caudal) across mice and treatment groups.

For immunofluorescent staining of floating brain sections, the blocking/permeabilization, washing, and antibody incubation steps were as described above for DAB staining, except the quenching step was omitted and secondary antibodies were Alexa Fluor conjugates (A488 or A568) (Thermo Fisher Scientific). For confocal experiments, sections were triple stained with Alexa Fluor 488-conjugated wheat germ agglutinin (A488WGA, Thermo Fisher Scientific), To-pro3 (Thermo Fisher Scientific), and antibody to TH, to distinguish plasma membrane, nuclei, and DA neurons, respectively, in the SN region. A488WGA and ToPro 3 were included during the secondary antibody incubation step. After mounting onto slides, sections were dried briefly (3 to 5 min) before cover-slipping with Fluoro-Gel (EMS) and sealing the coverslip with clear nail polish. Non-confocal fluorescent images were captured with either an Olympus IX70 inverted fluorescence microscope with an DP70 camera and DP Controller 2.1 software or a BX53 upright fluorescent scope with a DP72 camera and cellSens software (Olympus). ImageJ/Fiji was used to measure fluorescence intensity of TH staining; the striatal region was outlined and the mean intensity (range of 1-255) was determined, and mean background intensity (from adjacent unstained region) was subtracted. Confocal images were captured using a confocal laser scanning microscope (Zeiss LSM 510 meta) with Argon 488, DPSS-561, and HeNe633 laser lines, and Zeiss ZEN software (Carl Zeiss Microsopy, White Plains, NY. Confocal images were acquired by sequential capture for each channel, with the pinhole set to 1 airy unit and matched capture conditions for injected and non-injected counterparts within sections.

RNAseq. RNAseq was performed on RNA isolated from injected and non-injected SN regions harvested 3 weeks after stereotaxic injection of either AAV-hSYN1-miR-181 (1.65e10 vg; n=3 mice) or AAV-hSYN1-GFP (1e10 vg; N=5 mice). Total RNA samples isolated as described above were put over GeneJET RNA clean-up micro columns (cat# K0841, Thermo Fisher Scientific). RNA quality assessment and sequencing were performed by the Iowa Institute of Human Genetics, Genomics Division, at the University of Iowa. RNA quality and concentrations were determined with the Agilent 2100 Bioanalyzer, and Dropsense or Little Lunatic (Unchained Labs, Pleasanton, CA) reads. RIN values for samples were all >7.5. Illumina TruSeq® Stranded mRNA Library Prep kits (Illumina, San Diego, CA) were used to prepare sequencing libraries from mRNA, followed by reverse transcription to cDNA, fragment purification and ligation to indexed (barcoded) adaptors. Indexed libraries from AAV:miR-181 samples were prepared as a set and pooled for multiplex sequencing across two lanes on the Illumina HiSeq 4000 sequencer with 75-base paired-end reads. Indexed libraries from AAV:GFP samples were prepared as a set and pooled for multiplex sequencing across two lanes on the Illumina NovaSeq 6000 sequencer for 100 cycles with 50-base paired-end reads. RNA sequencing data was analyzed according to the Kallisto/Sleuth pipeline. Reads were aligned using Kallisto version 0.43.1, to Ensemble transcriptome release 98 (119215 transcript targets). The transcriptome was acquired on 30MAR2020 from “ftp://ftp.ensembl.org/pub/release-98/fasta/mus_musculus/cdna/Mus musculus.GRCm38.cdna.all.fa.gz”. Kallisto index and Kallisto quant were run using default settings, with specific input parameters of 100 bootstraps and 16 threads. The average mapping rate across samples was ˜87% and read depth was ˜15M reads per AAV-hSyn-GFP sample, and ˜40M reads per AAV-hSyn-miR-181 sample. Differential gene expression was quantified using Sleuth version 0.30.0, aggregating abundance on Ensemble genes, with a two-step Likelihood Ratio Test and Wald Test. Other packages include base R version 3.6.1, biomaRt version 2.42.1, dplyr version 0.8.5, ggplot2 version 3.2.1.

Gene ontology analyses were performed by inputting the indicated gene sets into the using ToppFun web-server tool, ToppGene Suite24 and using default settings. Results were exported and up to the top 50 enriched terms in each “Category” were tabulated.

Statistics. Statistical analyses were performed using available software and tools 9 e.g. Prism GraphPad and R package, with guidance from the University of Iowa Department of Biostatistics).

### EXAMPLE 2

### Modulation of MicroRNA Activities in Parkinson's Disease

During the past decade, microRNAs (miRNAs) have emerged as key post-transcriptional regulators of gene expression and have been implicated in a vast array of human diseases, including several neurological conditions. These small non-coding RNAs (˜22-nts) are excised from larger genome-derived transcripts and subsequently incorporated into Argonaute (Ago) proteins where they act predominantly to destabilize transcripts by base-pairing to target mRNAs. Recently, a transcriptome-wide map was generated of thousands of miRNA binding sites in human brain using Ago CLIP-seq (crosslinking immunoprecipitation coupled with high-throughput sequencing). CLIP-seq is comparable to CHIP-seq except that the method identifies RNA sequences complexed with RNA-binding proteins rather than DNA sequences complexed with DNA-binding proteins. Among the data, an interesting interaction between miR-181 and mRNA encoding tau was identified, which was later confirmed to be functional in in vitro experiments.

Tau exists as two major isoforms, known as 3R and 4R, which result from alternative splicing inclusion of exon 10. Previous studies have shown that tipping the balance in the cellular 3R:4R Tau ratio is a critical determinant in causing neuronal dysfunction. As such, tau has been implicated in a number of neurodegenerative diseases, including Alzheimer's (AD) and some forms of Parkinson's (PD) with dementia, in which abnormal neurofibrillary tangles composed of tau are observed. Interestingly, more recent PD biomarker studies have identified decreased levels of total tau in cerebrospinal fluid (CSF) from PD patients. This result surprised the field, raising many questions to be subject for future investigation. Given our developing tau:miR-181 story, we found this observation intriguing, especially in light of other studies noting differences in miR-181 levels in plasma and/or CSF samples in AD and PD patient samples. In particular, the Burgos et al. study revealed a supportive dichotomy between miR-181 in AD and PD; they found that CSF miR-181 levels inversely correlate with neurofibrillary tangles in AD, but positively correlate with Lewy bodies in PD (i.e. lower miR-181 associates with AD, whereas higher miR-181 is linked to PD). Together, these data point to a model where optimal neuron function might require a balance not only in miR-181 levels, but also tau, meaning that the dosing of drugs targeting these pathways could be subject to the “Goldilocks Principle”. The present studies specifically test whether modulation of miR-181 activity influences PD pathogenesis and disease outcomes in mice.

Based on earlier data, it was hypothesized that miR-181 is a key player in PD pathogenesis. In addition to the supporting empirical data, this premise is further backed by multiple lines of bioinformatic-based outcomes which reinforce a likely role of miR-181 in coordinating PD signaling networks. This hypothesis was studied in vivo, using AAV vectors to overexpress or suppress miR-181 in the substantia nigra (SN) of mice. First, the influence of miR-181 overexpression or inhibition on target mRNA and/or protein expression in the SN was validated. Second, the neurological impact on of miR-181 overexpression or inhibition in normal mice, as well as a mouse model for PD was studied.

The overall goal of the proposed work is to establish miR-181 as a key regulator of PD pathogenesis in mice. This work will assess the effects of modulating miR-181 activity in the SN of mice and identify key miR-181 targets relevant to PD biology. We will determine if miR-181 regulation of tau is conserved in mice. Importantly, our Aim 2 studies will demonstrate whether miR-181 modulation is sufficient to induce or modify PD-related phenotypes (e.g. dopaminergic neuron loss and motor deficits). Together, this work has the potential to highlight the miR-181 regulatory network as an avenue for future investigation of disease mechanism and drug development, and this could be further bolstered by continued testing of miR-181 modulation in other PD model systems.

### Characterization of AAV-Directed Modulation of miR-181 Activity in Mouse SN

MiR-181 is expressed throughout the brain in non-neuronal and neuronal cell types, including dopaminergic neurons. To the best of our knowledge, there have been no reports describing the overexpression nor inhibition of miR-181 in the mouse brain. We have extensive experience with modulating gene expression in mouse brain using recombinant adeno-associated virus (AAV) engineered to express U6 promoter-driven small non-coding inhibitory RNAs (e.g. gene-targeted short-hairpin RNAs and naturally occurring miRNAs). In addition to these RNAs, we have also begun constructing and working with expressed miRNA inhibitory molecules [e.g. tough decoys (TuDs)], which bind up endogenous miRNAs, preventing them from engaging their natural targets. Employing TuD technology is particularly useful when targeting miRNAs belonging to much larger families, which share common “seed” sequences, the critical determinant for targeting capacity. For example, the miR-181 family includes four members: miR-181a, -181b, -181c, and -181d, some of which are duplicated throughout the genome. Thus, the use of classical genetic knockout strategies poses a challenge for studying miRNAs of this nature, as knocking out the entire miR-181 family would require three targeted loci on different chromosomes, with each locus encoding two miR-181 family members. Furthermore, the broad expression of miR-181 throughout various tissues would warrant layering on additional genetic approaches to achieve brain-specific knockout for the sake of querying the relevance to PD. Given these hurdles, a viral-based approach offers a straightforward alternative, allowing for local injection into the targeted brain regions of interest, and the ability to transduce various brain cell-types (e.g., neurons) depending on the AAV serotype employed.

So far, we have constructed and tested more than 10 plasmid-based TuD constructs targeting various miRNA families, including miR-181. Notably, these plasmids are compatible with AAV production, done here at the University of Iowa Gene Transfer Vector Core. In our hands, these vectors are robust inhibitors of miRNA function in cell culture and in vivo. The latter is highlighted by our recent generation of transgenic mice expressing TuD for the miR-29 family; these mice exhibited profound neurological deficits (e.g. abnormal gait and limb clasping; data not shown), which is consistent with two prior reports citing ataxia-like phenotypes in mice following inhibition of miR-29 in brain. With respect to miR-181 specifically, our preliminary data indicate that our TuD-181 construct suppresses —60% of the silencing activity derived from our miR-181 overexpression vector. For this aim, we will determine the in vivo efficacy of overexpressing and inhibiting miR-181 in the SN of wild-type mice, and evaluate the downstream effects on the expression of tau and other potential miR-181 targets.

Methods. Evaluating miR-181 overexpression or inhibition in mouse SN following injection with AAV-miR181 or AAV-TuD181 vectors. The UI Vector Core will produce AAV serotype 2/5 for each vector, including controls (i.e., scrambled TuD, scrambled miRNA and GFP only; note, all viruses co-express CMV-driven eGFP reporter allowing for subsequent tracking of transduction efficiency and patterns). AAV2/5 is known to transduce various cell types in the SN, including tyrosine-hydroxylase-positive (TH+) dopaminergic neurons. Viruses (2u1 of 1e13 vg/ml titer) will be stereotaxically injected unilaterally into the SN of wild-type adult mice (8 weeks of age), as previously reported. The uninjected contralateral side will serve as an additional control for histological and molecular analyses. Ten animals per virus will be injected, five for molecular analyses and five for histology. Four weeks post-injection, mice will be sacrificed and the brains harvested. For molecular analyses, GFP-positive tissue from the SN and striatum will be isolated using a tissue punch; GFP-negative tissue will also be taken from the contralateral side. Tissue punches will be processed for total RNA and protein allowing for QCPR and western blot analyses to measure mRNA and protein levels of miR-181 targets (see below). MiR-181 levels will be quantified using commercially available QPCR assays designed to detect mature miR-181 transcripts. The transduction of the virus will be evaluated by histological examination of GFP expression and co-localization to various cell types immunolabeled with classical markers (e.g. NeuN, GFAP, Olig2, tyrosine hydroxylase, and VMAT). Qualitative analysis of miR-181 localization and levels will also be determined by in situ hybridization.

Determining the effects of miR-181 modulation on tau and other miR-181 target genes. To determine the functional impact of modulating miR-181 activity, we will assess protein and mRNA levels of several miR-181 targets, including known and computationally-predicted (TargetScan algorithm) targets having relevance to synaptic functions (e.g. MAPK, GluA2, GRM5, TRPC1, etc.). Selection of these targets is guided by bioinformatic intersection of three types of data: 1) miR-181 targeting data (e.g. our CLIP-seq data or TargetScan), 2) publically-available microarray data for miR-181 overexpression studies, and 3) gene expression changes observed in the SN of PD patients (e.g. Gene Expression Omnibus deposited datasets: GSE7621, GSE11968). As a natural extension of our MIFF RRIA, we will also evaluate whether endogenous mouse tau mRNA and protein levels respond to miR-181 modulation in vivo. Notably, mouse tau is indeed a computationally-predicted target of miR-181 (TargetScan), yet this potential regulatory interaction has not been tested or reported in the literature.

### Evaluation of the Neurological Impacts of Modulating miR-181 in SN in Mice

Changes in miR-181 levels have been linked to PD; however, whether alterations in miR-181 levels are sufficient to induce or modify PD phenotypes remains unknown. Although our previous data highlight tau as an interesting miR-181 target, we acknowledge that the potential pathogenic or protective impact of miR-181 on PD could be elicited through the concerted regulation of several target genes, consistent with how miRNAs are viewed as coordinators of complex biological responses. Here, our goal is to determine the phenotypic outcomes resulting from AAV-mediated miR-181 modulation in the SN in wildtype and PD mice, and to assess whether observed outcomes are tau-dependent using tau knockouts. Tests in wildtype mice will address whether miR-181 modulation is sufficient to induce neuronal cell loss and/or abnormal motor behavior. For our PD mice, we will utilize a model wherein PD is induced by introducing AAV overexpressing alpha-synuclein (MJFF reagent) into the SN, similar to several published reports. To query the potential influence of miR-181 activity on PD-related phenotypes in this model, we will employ viral co-delivery, to enhance or suppress miR-181 activity in the same cells that overexpress alpha-synuclein. We initially considered using transgenic Thy1-aSyn PD mice (readily available in the Narayanan Lab; U of Iowa); however, these mice express aSyn throughout most of the CNS, displaying neurological abnormalities beyond the SN. Here, the proposed co-delivery strategy circumvents this confound, offering a more straightforward approach for our initial proof-of-concept studies.

Methods. Assessing motor performance and histopathology in miR-181 modulated wildtype and aSyn-overexpressing mice. Eight-week old wildtype mice will be stereotaxically injected unilaterally with AAV-miR181, AAV-TuD181 or control viruses, either with or without AAV-aSyn (10 mice per group), as previously reported. At 8 and 16 weeks post-treatment, motor parameters will be measured by open-field test, rotarod, gait analysis, and amphetamine-induced circling, each capable of discerning PD and wildtype mice. Mice will subsequently be euthanized for molecular and histological analyses (as described above; 5 mice per group for each). We will characterize viral transduction, miR-181 levels, miR-181 target expression, aSyn overexpression and aggregation (western blot and histology), TH-positive immunoreactivity in SN and striatum, loss of TH-positive neurons (stereology), and inflammatory responses (microglial activation by Iba1 stain).

### EXAMPLE 3

### Modulation of MicroRNA Activities in Parkinson's disease

The in vivo influence of miR-181 over-expression or inhibition on target mRNA and/or protein expression in the SN was studied.

Assessment of first-generation AAV5-U6miR181b2/CMVeGFP and AAV5-TUD181/CMVeGFP vectors in vivo. At 8 weeks post unilateral injection into the SN/VTA of C57BL/6J mice, SN/VTA and striatal regions were dissected from injected and non-injected hemispheres and processed for RT-qPCR and western blot to determine the effects on computationally-predicted miR181 targets. We detected no significant changes in transcript abundance (6 targets tested) or protein abundance (3 tested) for either vector. Functional effects of miRNA are mediated by the fully processed, mature form, which is cleaved from a longer primary transcript. The mature miRNA sequence for miR181 is conserved across species, and is naturally abundant in the brain. After brain injection of AAV5-U6miR181b2, the vector-derived primary miR181 transcript (pri-miR181b) was readily measured in injected SN/VTA; however mature miR181b levels (sum of endogenous and vector-derived) were not significantly elevated above endogenous baseline level, signifying impaired transcript processing and thus a need for an improved miR181expression construct. For AAV5-U6TUD181/CMVeGFP, endogenous miR181b was reduced by 15%, demonstrating functionality of this construct in vivo; we still need to measure miR181a in these samples. Considering that the TuD functions primarily to sequester miRs and secondarily to promote some degradation, we are encouraged to see a significant reduction in miR-181b levels. Although this is not reflected in our qPCR and western blot data for potential miR-181 targets, our set of targets is limited, and there are many caveats to “cherry-picking” (i.e., guessing) miR-181 targets. Our lab specializes in using transcriptome-wide approaches to delineate empirical miR targets, and future studies should employ RNA-seq strategies to delineate responsive miR-181 targets in the mouse SN, in unbiased fashion, followed by confirmation at the protein level.

Conclusions: AAV-TuD-181 appears to suppress miR-181 levels in mouse SN, but the miR-181 overexpression vector failed to increase miR-181 and needs optimization. No miR-181 responsive targets have yet been identified in treated SN.

### Development of an Optimized Second-Generation AAV5-hSyn1miR181al/b2 Construct

To enhance expression level of miR181, we designed a new construct with Pol-II driven expression of a primary transcript encoding tandem miR181a1 and miR181b2 sequences. Pilot testing in culture using the CMV promoter showed specific knock-down by psi-check luciferase assay and strong miR181 expression, as compared to our first generation vector. This new expression cassette was sub-cloned into the AAV5-hSyn1 shuttle plasmid, which utilizes the neuronal-specific hSyn1 promoter. Injection of AAV5-hSyn1-miR181a1b2 unilaterally into mouse SN resulted in ˜5-fold increase in miR-181a levels (p=0.05) in SN and slight increase in striatal projections. miR-181b levels were similarly increased. Also, follow-up western blotting for ˜10 candidate miR-181 targets showed that none of those evaluated were significantly down-regulated in miR-181 overexpressing SN, though some may have been trending.

Conclusions: Optimization of the miR-181 overexpression AAV was successful; however, responsive miR-181 targets in mouse SN remain elusive. An unbiased approach to identifying targets (i.e. RNA-seq) is warranted, and such an experiment will be robust considering that we can compare both overexpression and inhibition of miR-181 to a common control.

### Evaluation of the Neurological Impacts of Modulating miR-181 in Mouse Brain (Normal and PD)

The neurological impact on of miR-181 overexpression or inhibition both in normal mice, and in a mouse model of PD was studied. In particular, a viral-based mouse model of PD was developed.

### Establishing an AAV-based PD Mouse Model

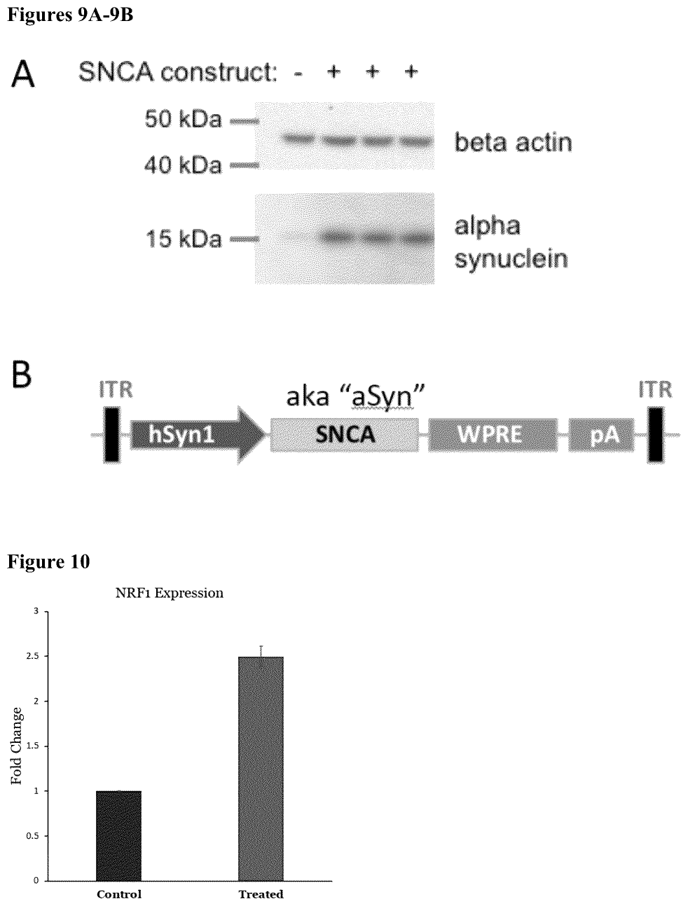

Early in this study, we obtained MJFF vector from UNC (AAV5-CBA-SNCAco) and injected two cohorts of mice with different doses. However we did not observe significant disease phenotypes. The current lack of a reliable vector-based rodent PD model is inline with the recent MJFF initiative to develop AAV-based models with consistent outcomes across labs. For our study, we moved forward with construction of an alpha synuclein expression cassette that combines elements reported by others to perform well in DA neurons of the mouse SN. Specifically, we incorporated the human synapsin 1 promoter (hSyn1), the wt human SNCA cDNA, and the WPRE cis element (FIG. 9B), and packaging into the AAV serotype 5 capsid, shown to transduce a high proportion of mouse SN neurons. We used the Addgene “hSyn-eGFP-WPRE” as our parent AAV shuttle plasmid, in which we replaced the eGFP with human SNCA. The wildtype human SNCA cDNA sequence was first tested for protein synthesis by western blot for alpha synuclein (FIG. 9A). The UI VVC produced AAV5-hSyn1-SNCA-WPRE vector and two initial cohorts of C57BL/6J mice have been injected at “high dose” (1.5×1010 VG) and “low dose” (3×109 VG), unilaterally into the VTA/SN. Representative mice from each group were sacrificed at 2 weeks post injection to assess transgene expression. Initial results from a high-dose injected mouse indicate strong expression of human alpha synuclein in the injected region, overlapping with the TH positive neurons of the SNpc. Human alpha synuclein positive neurites were also readily detected in the injected-side striatum. Other cohorts were sacrificed at 14.5 weeks post-injection for histological analyses, which revealed clear evidence that our AAV-hSyn1-SNCA vector induced robust degeneration of TH-positive neurons (relative to the uninjected control side), and this corresponded with the formation of prominent phospho-S129 aSyn aggregates and regional inflammation (increased mouse igG). Notably, this was not simply due to overexpressing any protein (e.g. some have reported that high-level GFP expression can induce toxicity), as our follow-up studies with the AAV-hSyn-GFP control vector show robust GFP expression that was well-tolerated in comparison to the AAV-hSynI-aSyn vector.

Conclusions: The MJFF vector from UNC (AAVS-CBA-SNCAco) did not work in our hands, so we generated a new AAVS-hSyn1-SNCA-WPRE vector that we found to mediate aSyn-induced PD-related pathology in mouse SN.

### Assessing the Effects of miR-181 Modulation in Normal Mouse SN

Published and unpublished data support that miR-181 levels are elevated in PD patient brains, and miR-181 has been shown to play a role in promoting cell death in various experimental settings. As part of this aim, we tested the long-term effects of miR-181 overexpression (optimized vector) or inhibition (TuD181) in mouse SN using our AAV vectors. For this, we compared miR-181 and TuD-181 treated brains to brains injected with either 6-OHDA or our AAV-SNCA vector, which both induce degeneration of TH-positive neurons. In this experiment, we also included lower doses of AAV-SNCA. Interestingly, we found that miR-181 overexpression induced loss of TH-positive staining in mouse SN (more so compared to a low dose of AAV-SNCA, 3x10e9 vg), whereas TuD-181 expression was well-tolerated.

Conclusions: miR-181 overexpression induced toxicity in mouse SN dopaminergic neurons to a greater degree than a lower dose of AAV-SNCA. It will be interesting to test lower doses of both vectors in combination to test synergistic effects (e.g does modest elevation of miR-181 exacerbate aSyn-induced pathogenesis at lower levels of aSyn overexpression?). Considering the TuD-181 vector was well-tolerated, we next tested if TuD-181 could block AAV-SNCA-induced pathogenesis.

### Evaluating the Effect of TuD-181 on AAV-SNCA-Induced Pathogenesis in Mouse SN

Mouse study of the effects of TuD181 on PD model, n=9-10 per group

Four groups; AAV vectors injected bilaterally into SN:

A) non-injected

B) AAV-hSyn1eGFP +AAV-U6-control

C) AAV-hSyn1-SNCA +AAV-U6-control

D) AAV-hSyn1-SNCA +AAV-U6-TuD181

Post-injection behavior/motor phenotypes:
Accelerating rotarod
Open Field for multiple ambulatory parameters (baseline and amphetamine-induced)
Gait analysis (paw-print)—still analyzing the collected data

Behavior: After 15 weeks post-injection, no obvious differences in behavior were observed in any group.

Pathology and molecular assays: Following behavior/motor assessment, five mice per group were sacrificed and brains harvested for sectioning and histological analyses. The remaining 4-5 mice per group were sacrificed to collect striatum and SN regions for protein and RNA analyses. As observed previously, AAV-hSyn1-SNCA treatment induced severe loss of TH-staining in both SN and striatal regions compared to non-injected controls, whereas the control AAV-Syn1-eGFP was well-tolerated, indicating that the observed toxicity is likely caused by aSyn overexpression. Surprisingly, AAV-SNCA-induced loss of TH was almost completely attenuated in mice that were co-injected with the TuD181 vector, supporting that miR-181 inhibition is protective.

Conclusions: Our AAV-Syn1-SNCA vector induces substantial loss of TH staining in a consistent manner and the TuD181 vector (i.e. miR-181 inhibition) blocks this, perhaps indicating that miR-181 is an attractive therapeutic target. These data are consistent with the notion that miR-181 promotes cell death.

In summary, the efficacy of AAV-based vectors that are capable of modulating miR-181 levels in mouse brain were generated and demonstrated. MiR-181 has been shown to be elevated in PD brains, and our data indicate that increased miR-181 levels may promote cell death in mouse SN. We have also generated and established our own AAV-based aSyn overexpression mouse model of PD. This model has robust neuropathology, and additional behavioral phenotypes need to be assessed. Most notably, we found that co-injection of AAV-TuD181 along with AAV-SNCA almost completely blocked AAV-SNCA-induced loss of TH staining in treated mouse SN.

### EXAMPLE 4

### Treating Dominant Rhodopsin Associated Retinitis Pigmentosa

Vision loss associated with the inherited retinal degenerative disorder retinitis pigmentosa (RP) results from death of the light sensing photoreceptor cells of the outer neural retina. Like the brain, the neural retina has little capacity to regenerate, and as a result, treatments are needed to prevent photoreceptor cell death in early-stage disease and replace photoreceptor cells in late-stage disease. Although collectively common, inherited retinal diseases are genetically heterogeneous such that patients with a disease caused by any given gene are rare in the general population. For example, in a study of 1000 consecutive families seen in our inherited eye disease clinic, disease causing mutations were identified in 104 different genes. Of the 760 families that were molecularly diagnosed in this study, 489 families had autosomal recessive disease, 172 families had autosomal dominant disease, 97 families had X-linked disease and 5 families had disease associated with mitochondrial mutations. The most prevalent of these 104 genes causes disease in about 1 in 10,000 people while the least common gene is estimated to cause disease in fewer than 200 people in the United States.

For decades, physicians and scientists have envisioned preventing vision loss in patients affected with inherited retinal diseases via some form of gene replacement therapy. The clinical success of AAV-mediated gene augmentation for the treatment of early stage RPE65-associated disease has been encouraging and suggests that this approach will also be useful for the treatment of many other recessive and X-linked disorders. However, this strategy is much less likely to be effective as a stand-alone treatment for patients with dominantly inherited diseases that result from toxic proteins (i.e., gain-of-function variants). Of the dominantly inherited genes reported in our 1000 families study, the gene encoding rhodopsin (RHO) was found to be the leading cause of photoreceptor degeneration. The evidence that heterozygous mutations in RHO act predominantly through a gain of function mechanism include the fact that there are a paucity of nonsense and frameshift mutations (i.e., the vast majority of patients diagnosed with dominant rhodopsin associated RP have a missense mutation). For example, in a study of 1000 consecutive families with inherited retinal disease, of the 34 patients who had rhodopsin associated RP, 31 harbored missense variants. Missense variants in the rhodopsin gene have been subdivided into 7 different classes based on functional consequences, the majority of which fall within class 2. Class 2 mutations such as Pro23His, the most common variant in our cohort, result in production of a misfolded protein, ER-stress/unfolded protein response (UPR) and death of rod photoreceptor cells.

Regardless of disease mechanism, patients with mutations in RHO typically present with night blindness and photoreceptor cell death beginning in the mid-peripheral retina, which gradually advances both anteriorly and posteriorly. Clinically, intra-retinal pigment deposition and narrowing of the inner retinal vasculature (which occurs in response to the overabundance of oxygen following the loss of oxygen consuming rods) are characteristic features of RP. While the central cone-rich macula can remain relatively spared until late in the disease, complete loss of rods typically results in the “innocent bystander” death of cones and eventual loss of high acuity vision. Although the exact cause of secondary cone cell death is debatable, loss of rod derived cone viability factor (RdCVF) and increased oxidative stress are believed to play a major role.

As indicated above, dominantly inherited rhodopsin-associated RP is caused by gain of function mutations that encode a toxic protein product. For this reason, standard gene augmentation alone is unlikely to be effective. In fact, overexpression of full-length rhodopsin has been attempted in transgenic Pro23His retinal degenerative mice and a complete lack of therapeutic efficacy was observed. For gene augmentation to be effective, simultaneous suppression of the toxic gene product is likely to be required. To that end, suppression strategies ranging from hammerhead ribozyme induced cleavage of rhodopsin mRNA to CRISPR-mediated deletion of the mutant rhodopsin allele have been attempted with varying degrees of success. One of the most promising knockdown and overexpression studies published to date demonstrated use of a single AAV to deliver both an shRNA that was shown to suppress rhodopsin in a mutation independent fashion and full-length rhodopsin that was altered to make it resistant to shRNA suppression. The authors reported excellent knockdown efficiency, with 30% transgene expression and mitigation of disease progression in a dog model for up to 8 months following treatment. While this dual knockdown and overexpression approach is promising, rod photoreceptor cells are quite sensitive to rhodopsin overexpression which has been shown to induce structural changes, disrupt normal photo response, and induce retinal degeneration. As a result, alternate methods designed to preserve photoreceptor cell structure and function needed. To this end, the use of novel gene-based treatment approaches to mitigate dominant rhodopsin associated RP in patient iPSC derived retinal organoids in vitro and rhodopsin mutant rats and pigs in vivo are being investigated. Specifically, a disease-mechanism-based strategy designed to mitigate ER-stress/UPR mediated photoreceptor cell death is being used to mitigate disease progression, which inhibits ER-stress induced rod cell death and mitigates disease progression via the novel miR-181 inhibitor, TuD-181.

### MiR181a/b in Rod Photoreceptor Cells are used to Reduce Mutant Rhodopsin Induced ER-Stress/UPR, Rod Cell Death and Secondary Loss of Cone Photoreceptor Cell Mediated Vision

Disease causing mutations in the rhodopsin gene have been subdivided into 7 different classes. Class 2 mutations such as Pro23His are the most common in the United States and result in a misfolded protein that gets retained in the ER. Retention of misfolded rhodopsin in the ER induces ER-stress and activation of the unfolded protein response (UPR) via release of GPR78/BiP from the ER receptors PERK, IRE1α, and ATF6. While the goal of the UPR is to alleviate ER-stress by restoring normal protein folding, if unsuccessful it results in activation of cell death pathways and rod photoreceptor cell degeneration. Enabling the UPR system to clear misfolded rhodopsin in residual rods may slow rod photoreceptor cell degeneration and in turn prevent secondary cone cell death and loss of high acuity vision. Several studies targeting different components of the UPR pathway have already been performed. Overexpression of GPR78/BiP in a cell line containing Pro23His rhodopsin reduced ER-stress and expression of the pro-apoptotic factors CHOP and caspase-7. Interestingly, aged Pro23His rats were found to have reduced levels of GPR78/BiP and increased levels of CHOP. AAV mediated overexpression of GPR78/BiP prevented ER-stress induced apoptosis, photoreceptor cell degeneration and loss of electro retinal function for up to 3 months following delivery. In a similar study, overexpression of the BiP co-chaperone and reductase Erdj5 in rod photoreceptor cells was shown to promote rhodopsin degradation, prevent photoreceptor cell death and slow the rate of visual decline in the Pro23His rat.

Several studies have implicated micro-RNAs as regulators of UPR and ER-stress induced neuronal cell death. For instance, it was found that following stroke, miR-181 becomes elevated in the ischemic core where cells degenerate and decreased in the penumbra where cells survive. In this model, miR-181 elevation repressed GRP78/BiP protein expression and increased the area of neural degeneration. Conversely, repression of miR-181 resulted in elevation of GRP78/BiP protein levels and protection of neurons against ischemia-induced apoptosis. More recently, the role of miR-181 and its effect on neuronal cell health has been expanded beyond ER-stress. For instance, as discussed above, it was recently demonstrated in a Parkinson's model that miR-181a/b target mRNAs are broadly downregulated in both aging and disease. RNA-seq revealed miR-181a/b mediated repression of genes involved in synaptic transmission, neurite outgrowth and mitochondrial respiration. AAV mediated overexpression of an inhibitor of miR-181a/b (TuD-181) was found to prevent degeneration of dopaminergic neurons effectively delaying disease progression. Recent studies have demonstrated a similar result in models of optic nerve injury and inherited retinal degeneration associated with disruption in mitochondrial biogenesis and function. Specifically, AAV mediated delivery of a miR-181 inhibitor was found to slow retinal ganglion cell death in a mouse model of Leber hereditary optic neuropathy and retinal degeneration in both the Pro347Ser rhodopsin and Pde6b null mouse models of RP.

The use of the novel miR-181a/b inhibitor TuD-181 to mitigate dominant Pro23His rhodopsin associated disease progression and preserve photoreceptor cell health and function is explored. By repressing miR-181 activity in rod photoreceptor cells the rate of rod and cone photoreceptor cell death is slowed in the models evaluated.

### Methods

### Cloning and Packaging of AAV5 Vectors Expressing miR181a/b Inhibitor

Transgene expression plasmids are generated carrying the PolIII human U6 promoter upstream of the miR181a/b tough decoy (TuD) sequence flanked by AAV2 ITR sequences. Transgene cassette plasmids are packaged into AAV vectors via triple transfection using the AAV5 capsid. AAV5 is used as it selectively target photoreceptor cells.

Assessment of vector toxicity and treatment efficacy in human retinal explants and patient iPSC-derived retinal organoids in vitro: To identify the optimal level of Tud-181 expression for mitigation of ER-stress without overexpression toxicity, human retinal explants and Pro23His patient iPSC-derived retinal organoids are used. Retinal explants from normal non-diseased human donor retina are also be generated and transduced. Human retinal explants and patient iPSC-derived retinal organoids are transduced at a multiplicity of infection (MOI) of 10⁴, 10⁵, or 10⁶ vg/cell. TuD-181 is delivered via AAV under control of the constitutive U6 promoter. An AAV vector carrying a scrambled control sequence (AAV5-U6-ctrl) is used as a control. Human retinal explants are harvested 1-week following transduction, while patient iPSC derived retinal organoids are harvested at 1-, 2-, 4-, 8-, and 12-weeks following transduction. Following harvest, immunocytochemistry and western blot analysis are performed using antibodies directed against photoreceptor cell-specific markers (including recoverin, rhodopsin, and ROM-1), ER stress/UPR markers (including GRP78/BiP, and CHOP), and apoptosis markers (including cleaved caspase 3, cleaved caspase 9, and cleaved PARP). TUNEL labeling is used to evaluate overexpression toxicity. To confirm that the miR-181 inhibitor was effective, total RNA is isolated using Trizol extraction and miR-181 and select miR-181 target gene expression are assessed using TaqMan assays for NRF1, COX11, GABRA1, KCNJ6, and CHCHD10, which are directly regulated by miR-181.

### Assessment of Vector Toxicity and Treatment Efficacy in the Pro23His Rat Model in Vivo

To determine if the AAV5-U6-TuD181 vector can preserve rod photoreceptor cells and in turn cones and cone cell function in vivo, the retinal degenerative Pro23His rhodopsin mutant rat model is used. Specifically, two groups of rats, one at 1-month of age (early disease prior to significant rod cell loss) and one at 3-months of age (with more advanced disease characterized by significant rod loss but retention of most of the cones), receive a single 10 μl subretinal injection of AAV5-U6-TuD. To determine the optimal dose that is both safe and maximally effective at suppression ER-stress in vivo, three separate doses are tested (i.e., 10⁸, 10⁹, 10¹⁰ vg/animal). A single subretinal injection of AAV5-U6-ctrl vector in the contralateral eye is used as a control. Animals are sacrificed 4-(n=12, 6M & 6F) and 12-(n=12, 6M & 6F) weeks post-injection (2 animal groups×3 AAV doses×2 timepoints×12 animals per timepoint (6 for each of 2 analysis modalities: immunocytochemistry and western blot)=144 rats total). Retinal structure and function are analyzed via fundus photography, OCT, and ERG at 2-, 4-, 8-, and 12-weeks following injection. Following OCT and ERG analysis the animals are sacrificed, and the eyes are enucleated, fixed, cryopreserved, and sectioned for immunohistochemical and western blot analyses using antibodies targeting the ER stress/UPR and apoptosis markers outlined in the in vitro experiments above. ONL layer preservation is evaluated by confocal imaging and spider plots will be generated. ONL layer preservation is evaluated by confocal imaging.

### Assessment of Vector Toxicity and Treatment Efficacy in the Pro23His Pig Model in Vivo

To determine if the AAV5-U6-TuD181 vector can preserve photoreceptor function in a large clinically relevant model system, retinal degenerative Pro23His rhodopsin mutant pigs are used. As described above, all subretinal injections are performed within the temporal retinal arcades. For these in vivo experiments, 12 non-immunosuppressed Pro23His rhodopsin mutant pigs are used. Specifically, 6 male and 6 female pigs, each 4 weeks of age (when most rod photoreceptor cells remain), receive a single 300 μl subretinal injection of AAV5-U6-TuD at the optimal dose determined in the in vivo rat experiments above. A single subretinal injection of empty AAV5-U6-ctrl vector in the contralateral eye is used as a control. Animals are sacrificed 4- and 12-weeks post-injection with interim clinical analysis (fundus imaging, OCT, and ERG) performed at 2- and 8-weeks. Subretinal injections are performed. In addition to the AAV5-U6-ctrl vector, non-injected peripheral retina is used as a “no injection” control. Retinal structure and function will be assessed via OCT and ERG. Following sacrifice, the injected area of retina is bisected and half is used for immunohistochemistry and half is used for western blot analysis. Transgene expression and ONL layer preservation are evaluated immunohistochemically. Western blot analysis is performed to evaluate expression of miR-181 targets (including NRF 1, COX11, GABRA1, KCNJ6, and CHCHD10), ER-stress/UPR markers (including GRP78/BiP, PERK, IREL ATF6 and CHOP) and apoptosis markers (including cleaved caspase 3, cleaved caspase 9, and cleaved PARP).

### Results

To demonstrate the ability to generate, package and deliver TuD-181, subretinal injections of AAV5-U6-TuD181 were performed in Sprague Dawley rats. For this experiment, the vector was engineered to include a GFP expression cassette independent of TuD-181 to confirm transduction. It was possible to readily detect cells transduced with the miR-181 inhibitor 4 weeks post-injection. No evidence of retinal toxicity was detected at the highest dose tested (i.e., 109 vg/animal). To demonstrate functionality of the miR-181 inhibitor following transduction qPCR analysis using probes targeted against the miR-181 target gene NRF 1 was performed. As shown in FIG. 10, overexpression of TuD-181 increased expression of NRF 1, indicating robust suppression of miR-181 in animals following subretinal injection of AAV5-U6-TuD-181.

Although the foregoing specification and examples fully disclose and enable the present invention, they are not intended to limit the scope of the invention, which is defined by the claims appended hereto.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description.

The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. An inhibitory molecule that inhibits miR-181.

2. The inhibitory molecule of claim 1, wherein the inhibitory molecule binds to miR-181a, miR-181b, miR-181c, and miR-181d.

3. The inhibitory molecule of claim 1, wherein the compound is RNA.

4. The inhibitory molecule of claim 3, wherein the RNA has at least 95% identity to SEQ ID NO:1 (TuD-181), SEQ ID NO:2, SEQ ID NO:3 or SEQ ID NO:4.

5. The inhibitory molecule of claim 3, wherein the RNA has at least 99% identity to SEQ ID NO:1 (TuD-181), SEQ ID NO:2, SEQ ID NO:3 or SEQ ID NO:4.

6. The inhibitory molecule of claim 3, wherein the RNA has 100% identity to SEQ ID NO:1 (TuD-181), SEQ ID NO:2, SEQ ID NO:3 or SEQ ID NO:4. 7 A nucleic acid encoding the inhibitory molecule of claim 1.

8. An expression cassette comprising the nucleic acid of claim 7 operably linked to a promoter.

9. The expression cassette of claim 8, wherein the promoter is a transiently expressed or is constitutively expressed.

10. The expression cassette of claim 8, wherein the promoter is a tissue-specific or inducible promoter.

11. The expression cassette of claim 8, wherein the promoter is a mouse U6 polIII promoter.

12. An microRNA inhibitor system comprising a nucleic acid vector and the expression cassette of claim 8.

13. The microRNA inhibitor system of claim 12, wherein the vector is an adeno-associated virus (AAV).

14. The microRNA inhibitor system of claim 13, wherein the vector is an AAV that specifically transduces neurons.

15. The microRNA inhibitor system of claim 14, wherein the AAV is AAV2/5, AAV2/1, AAV2/9, PHP.B, or PHP.eB.

16. The microRNA inhibitor system of claim 12, wherein the vector is plasmid.

17. A method of inhibiting miR-181 comprising contacting a cell with a therapeutic agent, wherein the therapeutic agent is selected from the group consisting of:
(a) an inhibitory molecule that inhibits miR-181, or
(b) the microRNA inhibitor system comprising a nucleic acid vector and an expression cassette, wherein the expression cassette comprises a nucleic acid encoding an inhibitory molecule that inhibits miR-181 operably linked to a promoter,
wherein the inhibitory molecule or microRNA inhibitor system reduces the level of target miR-181 by about 25% to 100%.

18. The method of claim 17, wherein the therapeutic agent reduces the level of target miR by about 90%.

19. A method of treating Parkinson's disease or retinal degeneration in a patient in need thereof, comprising administering a therapeutic agent to the patient, wherein the therapeutic agent is selected from the group consisting of:
(a) an inhibitory molecule that inhibits miR-181, or (b) the microRNA inhibitor system comprising a nucleic acid vector and an expression cassette, wherein the expression cassette comprises a nucleic acid encoding an inhibitory molecule that inhibits miR-181 operably linked to a promoter.

20. A method of claim 19, wherein the disease is Parkinson's disease.


SEQUENCE LISTING (7 sequences)

SEQ ID NO: 1 - RNA, 113 nt, synthetic construct
  source 1..113: mol_type = other RNA
gacggcctcgagactggcaactcaccgacaaagatgaatgttcaagtattctggtcacagaatacaaccttaccgacaaagatgaatgttaacactagtctcggggccgtctt

SEQ ID NO: 2 - RNA, 23 nt, synthetic construct
  source 1..23: mol_type = other RNA
actcaccgacaaagatgaatgtt

SEQ ID NO: 3 - RNA, 22 nt, synthetic construct
  source 1..22: mol_type = other RNA
cttaccgacaaagatgaatgtt

SEQ ID NO: 4 - RNA, 10 nt, synthetic construct
  source 1..10: mol_type = other RNA
gttgaatgtt

SEQ ID NO: 5 - DNA, 70 bp, synthetic construct
  source 1..70: mol_type = other DNA
ctcgaggtgaatgttaccttgaaatgctccaacctgaatgttaggtttgccgttgaatgttagcggccgc

SEQ ID NO: 6 - DNA, 111 bp, synthetic construct
  source 1..111: mol_type = other DNA
gacggcctcgagactggaacctacacaatgcagaaataccacaagtattctggtcacagaatacaaccatcacaatgcagaataaccacaactagtctcggggccgtcttt

SEQ ID NO: 7 - DNA, 113 bp, synthetic construct
  source 1..113: mol_type = other DNA
gacggcctcgagactggcaactcaccgacaaagatgaatgttcaagtattctggtcacagaatacaaccttaccgacaaagatgaatgttaacactagtctcggggccgtctt